(19) United States
(12) Patent Application Publication
(10) Pub. No.: US 2010/0076451 A1
(43) Pub. Date: Mar. 25, 2010

(54) RIGIDIZABLE SURGICAL INSTRUMENT

(75) Inventors: Andrew M. Zwolinski, Cincinnati, OH (US); David B. Griffith, Cincinnati, OH (US); David Stefanchik, Morrow, OH (US)

Correspondence Address: K&L GATES LLP, 535 SMITHFIELD STREET, PITTSBURGH, PA, 15222, US

(73) Assignee: Ethicon Endo-Surgery, Inc., Cincinnati, OH (US)

(21) Appl. No.: 12/234,425

(22) Filed: Sep. 19, 2008

(51) Int. Cl. A61B 17/94

(52) U.S. Cl. 606/113; 600/564; 600/104; 606/1; 606/205; 606/170

(57) ABSTRACT

A rigidizable surgical instrument comprises a rigidizable member, a first collapsible arm, a second collapsible arm, and a specimen retrieval bag for retrieving biological materials. The collapsible arms may be located at the distal end of the rigidizable member. The specimen retrieval bag may have an open end and a closed end, and may be configured to be retained upon the collapsible arms. The rigidizable member may include a rigidizing component to rigidize the rigidizable member when the state-change material or a stiffening element is in a rigid state. The rigidizable member may be rendered substantially rigid when the rigidizing component is actuated and the rigidizable member may be rendered substantially flexible when the rigidizing component is deactuated. A rigidizing mechanism for actuating the rigidizing component may include a vacuum. Various end-effectors may be provided in addition to the specimen retrieval bag.

### BACKGROUND

The present disclosure generally relates to medical devices and more particularly to medical devices and methods useful in endoscopic procedures.

Access to the abdominal cavity may, from time to time, be required for diagnostic and therapeutic endeavors for a variety of medical and surgical diseases. Historically, abdominal access has required a formal laparotomy to provide adequate exposure. Such procedures, which require incisions to be made in the abdomen, are not particularly well-suited for patients that may have extensive abdominal scarring from previous procedures, those persons who are morbidly obese, those individuals with abdominal wall infection, and those patients with diminished abdominal wall integrity, such as patients with burns and skin grafting. Other patients simply do not want to have a scar if it can be avoided.

Minimally invasive procedures are desirable because such procedures can reduce pain and provide relatively quick recovery times as compared with conventional open medical procedures. Many minimally invasive procedures are performed with an endoscope (including without limitation laparoscopes). Such procedures permit a physician to position, manipulate, and view medical instruments and accessories inside the patient through a small access opening in the patient's body. Laparoscopy is a term used to describe such an “endosurgical” approach using an endoscope (often a rigid laparoscope). In this type of procedure, accessory devices are often inserted into a patient through trocars placed through the body wall. The trocar must pass through several layers of overlapping tissue/muscle before reaching the abdominal cavity.

Still less invasive treatments include those that are performed through insertion of an endoscope through a natural body orifice to a treatment region. Examples of this approach include, but are not limited to, cholecystectomy, appendectomy, cystoscopy, hysteroscopy, esophagogastroduodenoscopy, and colonoscopy. Many of these procedures employ the use of a flexible endoscope during the procedure. Flexible endoscopes often have a flexible, steerable articulating section near the distal end that can be controlled by the user by utilizing controls at the proximal end. Minimally invasive therapeutic procedures to treat diseased tissue by introducing medical instruments to a tissue treatment region through a natural opening of the patient are known as Natural Orifice Translumenal Endoscopic Surgery (NOTES)™.

Some flexible endoscopes are relatively small (about 1 mm to 3 mm in diameter), and may have no integral accessory channel (also called biopsy channels or working channels). Other flexible endoscopes, including gastroscopes and colonoscopes, have integral working channels having a diameter of about 2.0 mm to 3.5 mm for the purpose of introducing and removing medical devices and other accessory devices to perform diagnosis or therapy within the patient. As a result, the accessory devices used by a physician can be limited in size by the diameter of the accessory channel of the scope used. Additionally, the physician may be limited to a single accessory device when using the standard endoscope having one working channel.

Certain specialized endoscopes are available, such as large working channel endoscopes having a working channel of about 5 mm in diameter, which can be used to pass relatively large accessories, or to provide capability to suction large blood clots. Other specialized endoscopes include those having two or more working channels. One disadvantages of such large diameter/multiple working channel endoscopes can be that such devices can be relatively expensive. Further, such large diameter/multiple working channel endoscopes can have an outer diameter that makes the endoscope relatively stiff, or otherwise difficult to intubate.

The above mentioned minimally invasive surgical procedures have changed some of the major open surgical procedures such as gall bladder removal, or a cholecystectomy, to simple outpatient surgery. Consequently, the patient's recovery time has changed from weeks to days. These types of surgeries are often used for repairing defects or for the removal of diseased tissue or organs from areas of the body such as the abdominal cavity.

One shortcoming associated with such minimally invasive surgical procedures is the removal of excised tissue through an opening in the body of a patient. When an infected specimen, such as an infected gall bladder or appendix, is removed, the surgeon must be extremely careful not to spill the infected contents of the specimen into the peritoneal cavity of the patient. A time-honored solution is the manual cutting of the large tissue mass into small pieces that can fit through the incision. However, with this process, fragments of tissue can be dropped and fluids can be spilled into the peritoneal cavity. This can be serious if the excised tissue is cancerous or infected as this can lead to the seeding and re-spreading of cancer or the spreading of the infection to healthy tissue.

Additionally, many current laparoscopic and endoscopic devices utilize articulating end-effectors to provide the user with more control over the orientation of the working end of the instrument. Integration of the controls for articulating, as well as actuating, a working end of a laparoscopic or endoscopic device tend to be complicated by the size constraints of the relatively small pathway through which it is inserted. The controls for an endoscopic device are further complicated by the flexibility of the shaft. Generally, the control motions are all transferred through the shaft as longitudinal translations, which can interfere with the flexibility of the shaft. There is also a desire to lower the force necessary to articulate and/or actuate the working end to a level that all or a great majority of surgeons can handle. One known solution to lower the force-to-fire is to use electrical motors. However, surgeons typically prefer to experience feedback from the working end to assure proper operation of the end effector. The user-feedback effects are not suitably realizable in present motor-driven devices.

Consequently, what is needed is an improvement over the above. The foregoing discussion is intended only to illustrate some of the shortcomings present in the field of the invention at the time, and should not be taken as a disavowal of claim scope.

### BRIEF DESCRIPTION OF THE FIGURES

The novel features of the various embodiments are set forth with particularity in the appended claims. The various embodiments, however, both as to organization and methods of operation, may best be understood by reference to the following description, taken in conjunction with the accompanying drawings as follows.

FIG. 1 is a diagrammatical view illustrating the use of one embodiment of an endoscope and a rigidizable surgical instrument according to the present invention inserted through a patient's mouth and esophagus to perform a cholecystectomy through the stomach wall.

FIG. 2A is partial perspective view of one embodiment of a portion of an endoscope inserted through an overtube; the endoscope substantially fills the circular cross-sectional area of the overtube.

FIG. 2B is a partial perspective view of another embodiment of a portion of an endoscope inserted through an overtube and an insertion portion of a rigidizable surgical instrument also inserted through the overtube and adjacent to the endoscope; the endoscope does not substantially fill the circular cross-sectional area of the overtube.

FIG. 3A is a perspective view of an embodiment of a rigidizable surgical instrument functioning as a rigidizable specimen retrieval device with an end-effector having a specimen retrieval bag for use with a rigidizable member according to the present invention; the rigidizable specimen retrieval device is shown in an unfired position.

FIG. 3B illustrates one embodiment of the rigidizable specimen retrieval device shown in a fired position.

FIG. 4A illustrates one embodiment of a proximal handle and a distal handle of the rigidizable specimen retrieval device in the unfired position.

FIG. 4B illustrates one embodiment of the distal end of the rigidizable specimen retrieval device in the unfired position.

FIG. 5A illustrates one embodiment of the proximal handle and the distal handle of the rigidizable specimen retrieval device in the fired position.

FIG. 5B illustrates one embodiment of the distal end of the rigidizable specimen retrieval device in the fired position.

FIG. 5C illustrates a close-up view of one embodiment of the distal end of the rigidizable specimen retrieval device in the fired position.

FIG. 6 illustrates one embodiment of the proximal end of the rigidizable specimen retrieval device with an outer sheath removed.

FIG. 7 illustrates one embodiment of the rigidizable specimen retrieval device shown in a manipulated position.

FIG. 8 illustrates a partial sectional view of one embodiment of a rigidizable member taken along the longitudinal axis with a tension wire extending through a central bore.

FIG. 9 illustrates one embodiment of the rigidizable specimen retrieval device in another manipulated position.

FIG. 10A illustrates one embodiment of a specimen retrieval bag when the rigidizable specimen retrieval device is in the fired and yet another manipulated position.

FIG. 10B illustrates a side view of one embodiment of the specimen retrieval bag.

FIG. 111 illustrates a close-up view of one embodiment of the proximal end of the proximal handle of the specimen retrieval device.

FIG. 12A illustrates one embodiment of the proximal handle and the distal handle of the specimen retrieval device retracting the rigidizable member and the collapsible arms.

FIG. 12B illustrates one embodiment of the distal end of the specimen retrieval device retracting the rigidizable member and the collapsible arms.

FIG. 12C illustrates a close-up view of one embodiment of the distal end of the specimen retrieval device retracting the rigidizable member and the collapsible arms.

FIG. 13A illustrates one embodiment of a knot pusher.

FIG. 13B illustrates one embodiment of the knot pusher interacting with an outer sheath of the specimen retrieval device.

FIG. 13C further illustrates one embodiment of the knot pusher interacting with an outer sheath of the specimen retrieval device.

FIG. 14A illustrates a partial sectional view of one embodiment of a rigidizable member taken along the longitudinal axis with a state-change material provided in a central bore.

FIG. 14B is an enlargement of one embodiment of a state-change material that may be introduced into the central bore for the purpose of rigidizing the rigidizable member.

FIG. 15A illustrates a partial sectional view of one embodiment of a rigidizable member taken along the longitudinal axis with a combination of a tension wire and a state-change material provided in a central bore.

FIG. 15B illustrates a partial sectional view of one embodiment of a rigidizable member taken along the longitudinal axis with a tension wire extending through a central bore and a flexible membrane provided over the length of the rigidizable member.

FIG. 16A is a cross-sectional view of one embodiment of a system including an endoscope inserted through an overtube and a portion of a rigidizable surgical instrument also inserted through the overtube and adjacent to the endoscope.

FIG. 16B is a cross-sectional view of another embodiment of a system including an endoscope inserted through an overtube and a portion of a rigidizable surgical instrument also inserted through the overtube and adjacent to the endoscope.

FIG. 17 illustrates one method of employing one embodiment of a rigidizable specimen retrieval device and an endoscope through an overtube to perform a cholecystectomy.

FIG. 18 illustrates another method of employing one embodiment of a rigidizable specimen retrieval device and an endoscope through an overtube to perform a oophorectomy.

FIG. 19 illustrates one embodiment of the rigidizable specimen retrieval device.

FIG. 20A is a perspective view of one embodiment of an end-effector having grasper jaws for use with a rigidizable member.

FIG. 20B is a perspective view of one embodiment of an end-effector having opposed biopsy jaws and a spike for use with a rigidizable member.

FIG. 20C is a perspective view of one embodiment of an end-effector having a snare loop for use with a rigidizable member.

FIG. 20D is a perspective view of one embodiment of an end-effector having scissors for use with a rigidizable member.

FIG. 20E is a perspective view of one embodiment of an end-effector having a needle knife for use with a rigidizable member.

FIG. 20F is a perspective view of one embodiment of an end-effector having a sphincterotome for use with a rigidizable member.

FIG. 20G is a perspective view of one embodiment of an end-effector having a hook knife for use with a rigidizable member.

Corresponding reference characters indicate corresponding parts throughout the several views. The various illustrated embodiments have been chosen for the convenience of the reader and not to limit the scope of the appended claims.

### DETAILED DESCRIPTION

Before explaining the various embodiments in detail, it should be noted that the embodiments are not limited in their application or use to the details of construction and arrangement of parts illustrated in the accompanying drawings and description. The illustrative embodiments may be implemented or incorporated in other embodiments, variations and modifications, and may be practiced or carried out in various ways. For example, the various end-effectors, including the specimen retrieval device and the specimen retrieval bag, disclosed below are illustrative only and not meant to limit the scope or application thereof. Furthermore, unless otherwise indicated, the terms and expressions employed herein have been chosen for the purpose of describing the illustrative embodiments for the convenience of the reader and are not to limit the scope thereof.

The various embodiments described herein are directed to medical devices and more particularly to devices and methods useful in minimally invasive endoscopic procedures. The various embodiments provide methods and devices useful with various medical procedures, including without limitation methods and devices useful with endoscopes, methods and devices employed through naturally occurring body orifices, and methods and devices related to the placement and positioning of endoscopic surgical tools. For example, in one embodiment, a surgical instrument can be used to effectively remove diseased tissue from an operating area; the surgical instrument may utilize a specimen retrieval bag to remove biological materials from a patient in a substantially sterile manner. Biological materials may be able to be removed in a more sterile manner through the use of a specimen retrieval bag which has sufficient volume to receive the biological material (e.g., gall bladder, ovary, fallopian tube, appendix). Embodiments of the surgical instrument enable an end-effector, such as the specimen retrieval bag mentioned above, to be manipulated by the endoscope and then locked into position to facilitate maintenance of the end-effector's proximity to the surgical target. A variety of different end-effectors are disclosed which may be useful for both endoscopic and laparoscopic applications. In one embodiment, an end-effector may be employed through a patient's natural orifice for performing a variety of surgical operations at various angles and positions. These and other embodiments are now illustrated and described with reference to the following figures.

Certain embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting embodiments and that the scope of the various embodiments is defined solely by the claims. The features illustrated or described in connection with one embodiment may be combined with the features of other embodiments. Such modifications and variations are intended to be included within the scope of the claims.

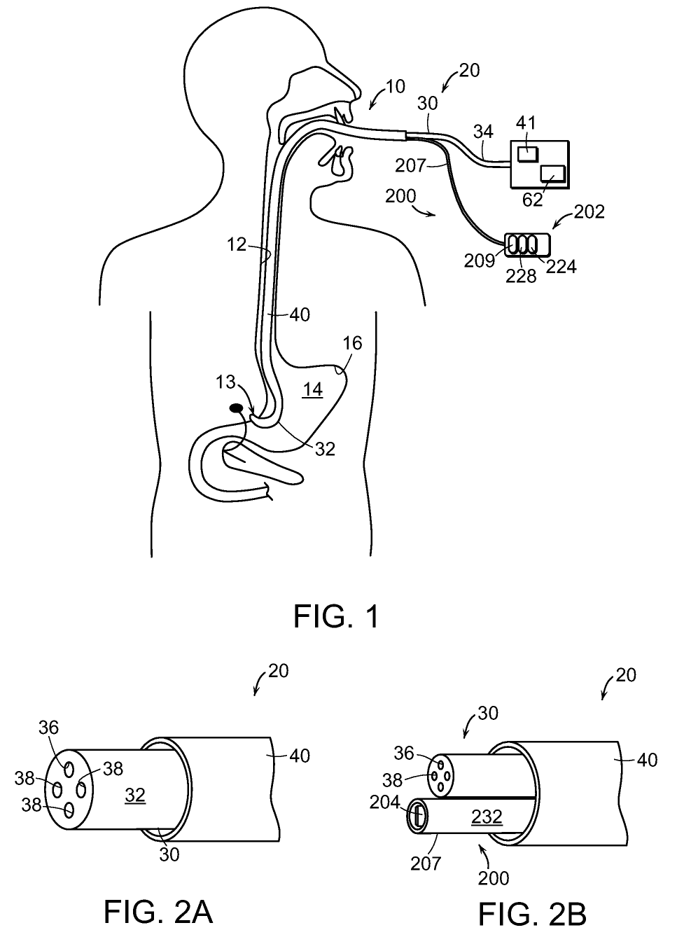

FIG. 1 is a diagrammatical view illustrating the use of one embodiment of a surgical instrument inserted through a patient's mouth and esophagus to perform a surgical activity such as to remove the patient's gall bladder, or perform a cholecystectomy, through the stomach wall. As illustrated in FIG. 1, in general form, a surgical instrument 20 is inserted through a natural orifice to form an opening through the stomach wall 16. The insertion may occur trans-orally (as depicted in FIG. 1), trans-anally, and/or trans-vaginally. In the example depicted in FIG. 1, the instrument 20 is inserted through the mouth 10 and esophagus 12 and into the stomach 14 to form an opening 13 through the stomach wall 16. In various embodiments, the instrument 20 may comprise a tubular member sized to receive an endoscope, a rigidizable surgical instrument, and/or any other suitable surgical device.

In various embodiments, for example, the tubular member may comprise a substantially hollow overtube 40. A flexible endoscope 30 may be inserted through the overtube 40 that is inserted into the stomach 14 through the patient's mouth 10. Additionally, a rigidizable surgical instrument 200 may be inserted through the overtube, but adjacent to the endoscope. Alternatively, the rigidizable surgical instrument 200 may be inserted through the endoscope, as well as the overtube 40. The surgical instrument 20 may be equipped with a variety of end-effectors, for example, a specimen retrieval bag and related components. FIG. 2A is partial perspective view of one embodiment showing a portion of the flexible endoscope 30 inserted through the overtube 40. In this embodiment, the endoscope 30 substantially fills the circular cross-sectional area of the overtube 40. A variety of different types of endoscopes are known and, therefore, their specific construction and operation will not be discussed in great detail herein. In various embodiments, the flexible endoscope 30 has a distal end 32 and a proximal end 34 and may operably support a video camera 36 that communicates with a video display unit 41 that can be viewed by the surgeon during the operation. The flexible endoscope 30 may comprise one or more working channels 38 extending therethrough for receiving various types of surgical instruments. FIG. 2B is a partial perspective view of another embodiment of a distal portion of an endoscope inserted through the overtube 40 and the distal end 232 of an insertion portion 207 of one embodiment of a rigidizable surgical instrument 200 also inserted through the overtube 40 and adjacent to the endoscope 30. In this embodiment, the endoscope 30 does not substantially fill the circular cross-sectional area of the overtube 40 and the rigidizable surgical instrument 200 is inserted between the overtube 40 and the endoscope 30. The rigidizable surgical instrument 200 includes an end-effector 204 for performing a surgical task, such as specimen retrieval. Further, at least part of the rigidizable surgical instrument 200 is capable of becoming rigid or stiff upon actuation from an appropriate mechanism, a rigidizing actuator 224. As shown in FIG. 1, the rigidizable surgical instrument 200 also includes a control device 202 that provides at least one control for each of the end-effector 204 and rigidizing actuator 224. The end-effector 204 may have several functions and therefore there is at least one end-effector control 209. Additionally, the control device 202 includes a rigidizing control 228 for controlling the rigidizing actuator 224.

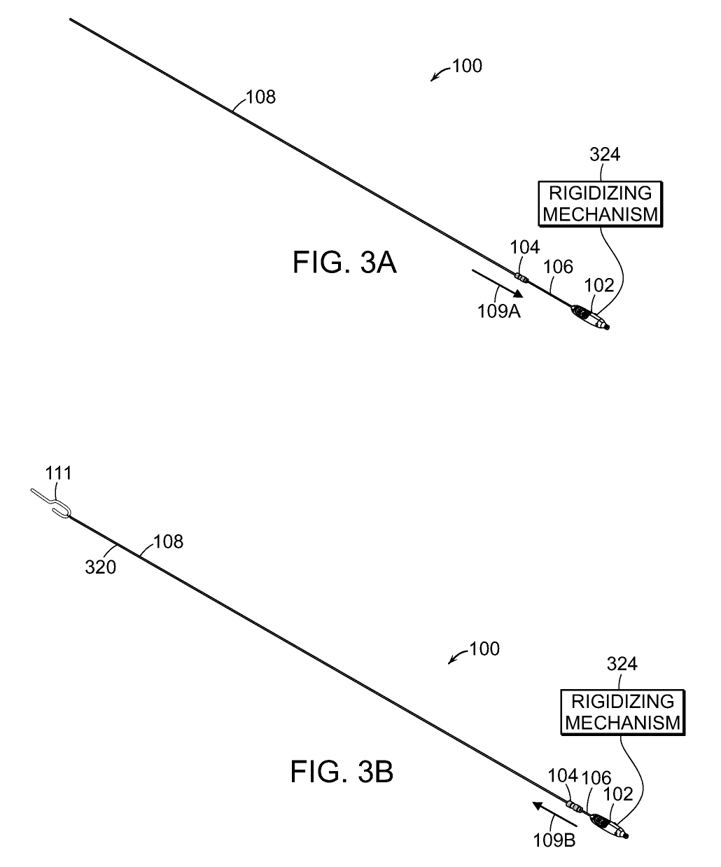

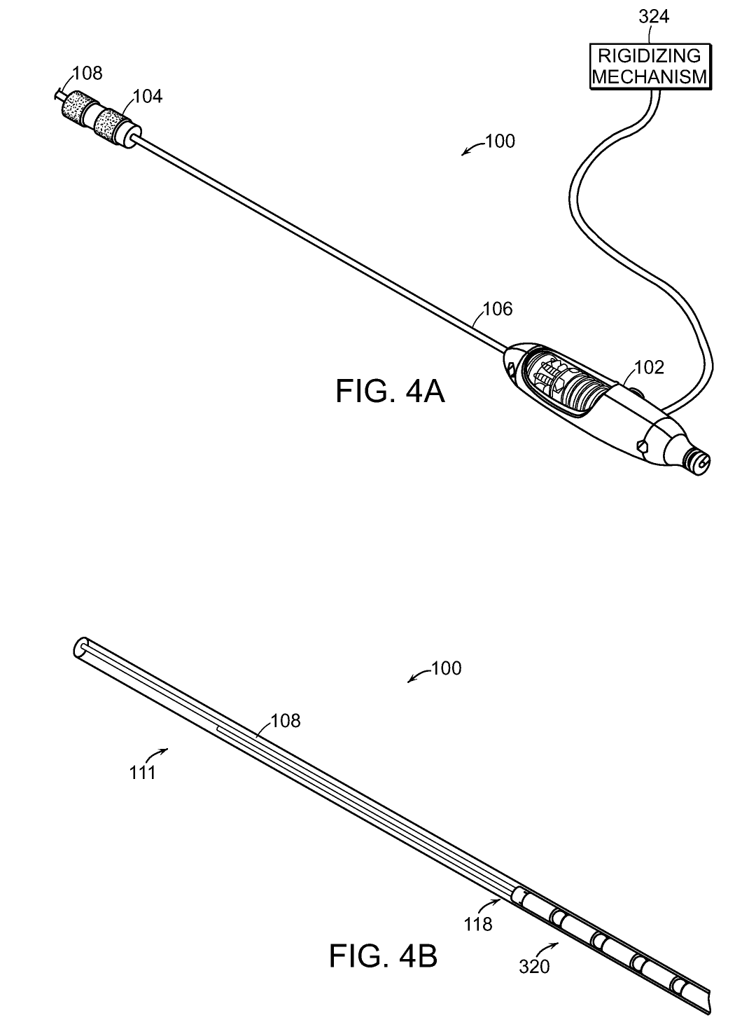

In at least one embodiment, a rigidizable surgical instrument may be configured as a specimen retrieval device by providing at least a specimen retrieval bag and related components as the end-effector. In this embodiment, the flexible endoscope 30 along with a specimen retrieval device 100 (FIGS. 3A, 3B, 4A, 4B, for example) may be used in minimally invasive surgical procedures. The specimen retrieval device 100 may be used in the removal of biological materials such as a gall bladder, ovaries, fallopian tubes, an appendix, or any other suitable material. For example, the specimen retrieval device 100 may be employed in a cholecystectomy to remove the patient's gall bladder. Cholecystectomies have traditionally been performed using laparoscopic techniques, or more invasive procedures such as an open cholecystectomy. A laparoscopic cholecystectomy requires several small incisions in the abdomen to allow the insertion of surgical instruments and a small video camera. After the incisions are made, the surgeon will inflate the peritoneal cavity with carbon dioxide or some other similar gas. The surgeon watches the video output provided by the video camera 36, for example, on the video display unit 41, and performs the gall bladder removal by manipulating the surgical instruments through the small incisions. An open cholecystectomy is a major abdominal surgery in which the surgeon removes the gall bladder through an incision which can range from about 10 cm to 20 cm. The patient's recovery time after an open cholecystectomy is quite long given the large incision in the abdominal cavity.

Newer procedures have developed which may be even less invasive than the laparoscopic procedures used in earlier surgical procedures. Many of these procedures employ the use of a flexible endoscope, such as the flexible endoscope 30, during the procedure. Flexible endoscopes often have a flexible, steerable articulating section near the distal end that can be controlled by the user by utilizing controls at the proximal end. As previously mentioned, minimally invasive therapeutic procedures to treat diseased tissue by introducing medical instruments to a tissue treatment region through a natural opening of the patient are known as NOTES™. NOTES™ is a surgical technique whereby operations can be performed trans-orally (as depicted in FIG. 1), trans-anally, and/or trans-vaginally.

The endoscope 30 comprises a substantially flexible shaft and can be any commercially available endoscope, such as a gastroscope or colonoscope having an articulating distal section, including a viewing element (e.g., the video camera 36) and a working channel (e.g., the working channel 38) at the distal end thereof. Any suitable endoscope, including without limitation gastroscopes and pediatric colonscopes can be used with various embodiments of the surgical instrument 20. Suitable endoscopes for use with the present invention include, without limitation, model PCF100, PCF130L, PCF140L, or PCF160AL endoscopes manufactured by Olympus Corporation of Japan. The overtube 40 can be sized and adapted to receive various diameter rigidizable surgical instruments and endoscopes, such as, but not limited to, endoscopes having a diameter from about 9 mm to about 14 mm. To introduce the endoscope 30 along side the specimen retrieval device 100 (FIGS. 3A, 3B, 4A, 4B) into a patient, the operator may start with a clean dry endoscope and a clean dry specimen retrieval device. The endoscope 30 also may include an adjustable portion, such as, for example, a steerable articulating section near the distal end 32 thereof. The steerable, articulating, or adjustable portion of the endoscope 30 is usually the distal 12 cm to 16 cm of the endoscope.

FIG. 3A is a perspective view of an embodiment of a rigidizable surgical instrument with an end-effector having a specimen retrieval bag for use with a rigidizable member. The rigidizable specimen retrieval device is shown in an unfired position. The specimen retrieval device 100 may comprise a proximal handle 102 and a distal handle 104. The specimen retrieval device 100 may further comprise a shaft assembly 106 and an outer sheath 108. A rigidizing mechanism 324 may be coupled to the proximal handle 102. In the unfired position, the specimen retrieval device 100 may be inserted into the substantially hollow overtube 40 alongside the flexible endoscope 30 such that the specimen retrieval device 100 is not inserted into any of the working channels 38 of the endoscope 30 (FIGS. 1 and 2B). Alternatively, the unfired specimen retrieval device 100 may be inserted into one of the working channels 38 of the flexible endoscope 30 (FIG. 2A). In the unfired position, the distal handle 104 is located distally from the proximal handle 102. To fire the specimen retrieval device 100, the distal handle 104 may be translated proximally towards the proximal handle 102 in the direction indicated by arrow 109A in FIG. 3A.

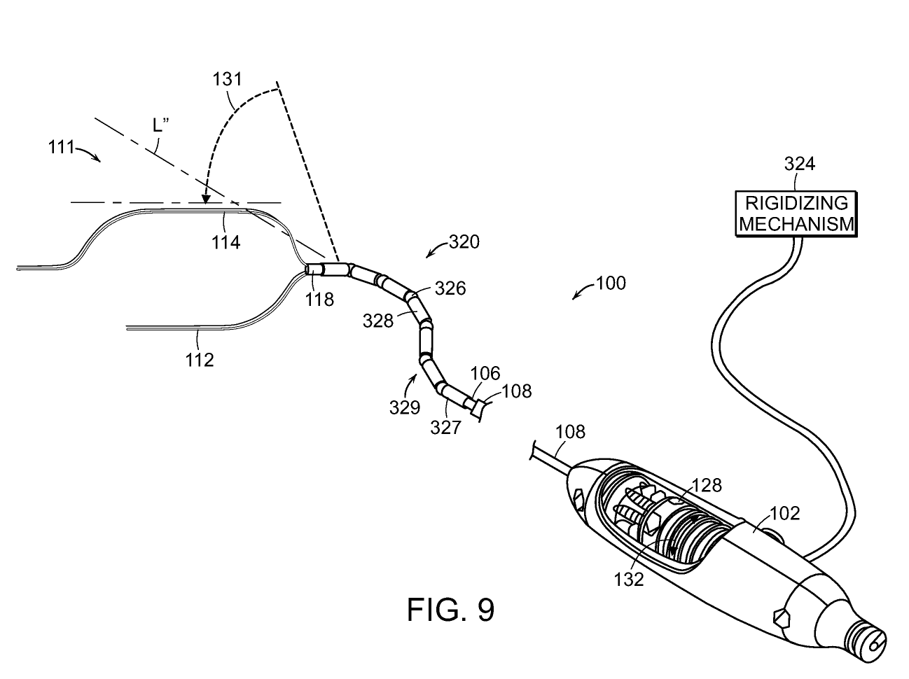

It will be appreciated that the terms “proximal” and “distal” are used herein with reference to a clinician gripping the proximal handle 102 of the specimen retrieval device 100. Thus, the specimen retrieval bag 110 (FIG. 9A) is distal with respect to the handle assemblies of the specimen retrieval device 100. It will be further appreciated that, for convenience and clarity, spatial terms such as “top” and “bottom” also are used herein with respect to the clinician gripping the proximal handle 102. However, surgical instruments are used in many orientations and positions, and these terms are not intended to be limiting and absolute.

FIG. 3B illustrates one embodiment of the specimen retrieval device 100 shown in a fired position. In the fired position, the specimen retrieval device 100 deploys an end-effector that may include a collapsible arm assembly 111 which may be configured to retain a specimen retrieval bag 110 (illustrated in FIG. 9A) for removing the biological material. In the fired position, the distal handle 104 is located proximally to the proximal handle 102. To return the specimen retrieval device 100 to the unfired position, the distal handle 104 may be translated distally away from the proximal handle in the direction indicated by arrow 109B in FIG. 3B. In the fired position, a portion of a rigidizable member 320 may be exposed that enables positioning, placing, and angling of the end-effector. The rigidizing mechanism 324 is further coupled to the rigidizable member 320 such that the rigidizable member 320 may be rigidized by the rigidizing mechanism 324 when appropriate, and is described in more detail below.

FIG. 4A illustrates one embodiment of the proximal handle 102 and the distal handle 104 of the specimen retrieval device 100 shown in the unfired position. FIG. 4B illustrates one embodiment of the distal end of the specimen retrieval device 100 shown in the unfired position. The sheath 108 is shown as transparent in FIG. 4B to visualize the components internal to the sheath in the unfired position. In the unfired position, the outer sheath 108 may contain at least the arm assembly 111, a rigidizable member 320, a knot pusher 118, and a specimen retrieval bag 110 (FIG. 9A). The outer sheath 108 may be connected to the distal handle 104 through any suitable fastening means which may include fusing, welding, gluing, bolting, riveting and/or screwing, for example. The assembly of the outer sheath 108 and the distal handle 104 may be configured to be received by the shaft assembly 106. Further, the shaft assembly 106 may internally include a coupling (not shown), which couples the rigidizable member 320 to the rigidizing mechanism 324 via the proximal handle 102.

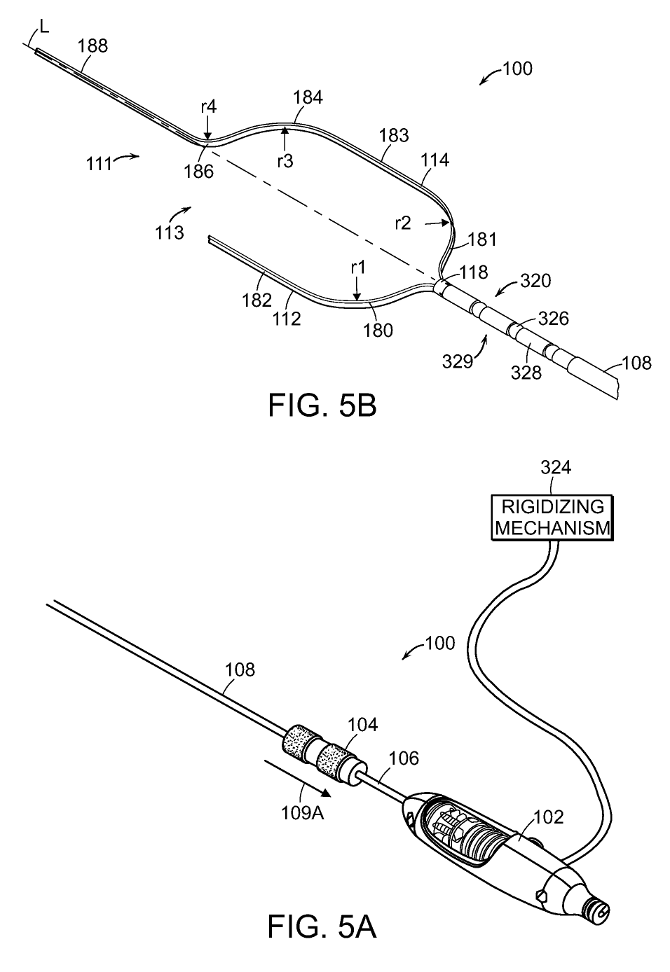

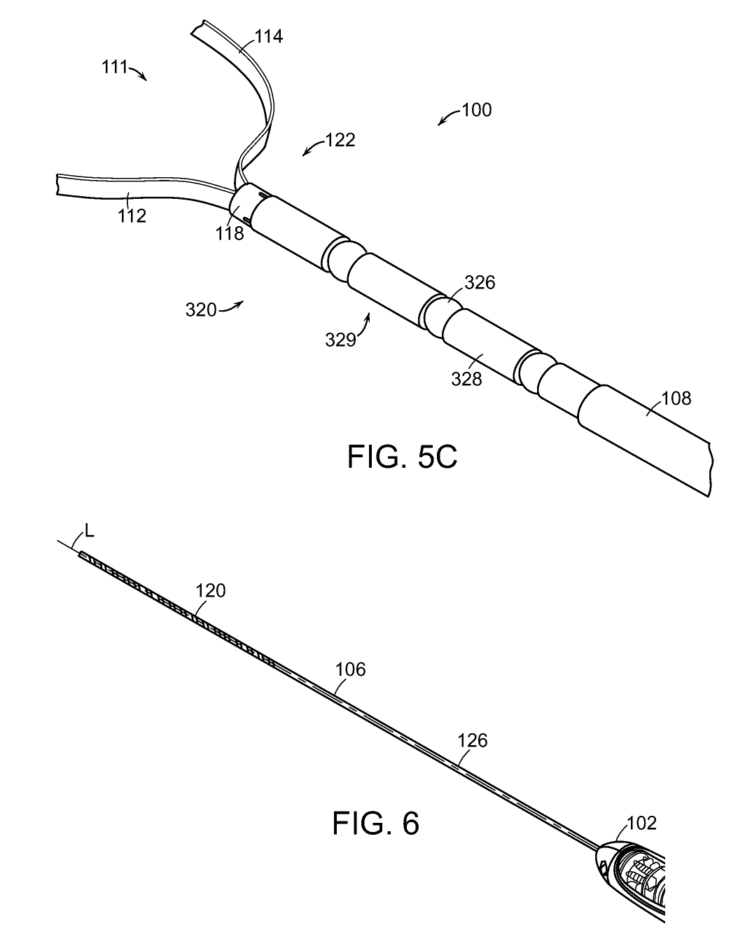

FIG. 5A illustrates the location of the proximal handle 102 relative to the distal handle 104 of the specimen retrieval device 100 in the fired position. FIG. 5B illustrates the distal end of the specimen retrieval device 100 in the fired position. The distal handle 104 may be translated proximally towards the proximal handle 102, as shown by arrow 109A in FIG. 5A, to expose the specimen retrieval bag 110 (FIG. 9A), the arm assembly 111, the knot pusher 118, and at least a portion of the rigidizable member 320. In various embodiments, the rigidizable member 320 may comprise multiple assemblies 329, each comprising a ball 326 and a socket 328; these are described in more detail below.

In various embodiments, the arm assembly 111 may comprise a first collapsible arm 112 and a second collapsible arm 114. The first collapsible arm 112 and the second collapsible arm 114 may be fabricated from a resilient material such as a resilient metal, or plastic, or any other suitable resilient material. This resilient material may cause the arms 112, 114 to “spring” to an open position once they are exposed and removed from forces created by an inner wall of the outer sheath 108. The resilient material may allow the arms 112, 114 to return to a substantially straight “collapsed” position once they are retracted into the outer sheath 108 (see FIG. 11B, for example). Further, after the rigidizable member 320 has been manipulated to a non-straight configuration as described herein, the resilient material may force the rigidizable member 320 to return to a substantially straight position as it is retracted into the outer sheath 108. The exposure and retraction of the arms 112,114 and the rigidizable member 320 may be repeated on numerous occasions.

In various embodiments, the first collapsible arm 112 and the second collapsible arm 114 may extend distally from the rigidizable member 320 along an axis L. The first collapsible arm 112 and the second collapsible arm 114 may define an opening 113 therebetween. In at least one embodiment, the first collapsible arm 112 may be asymmetric to the second collapsible arm 114. In various embodiments, the first collapsible arm 112 may comprise an arcuate portion 180 and a substantially straight portion 182. In the open position, the arcuate portion 180 of the first collapsible arm 112 may be defined by a radius “r₁.” In the open position, the substantially straight portion 182 may be formed in a straight section, an elliptical section, a circular section, or any other suitable shaped configuration.

In various embodiments, the second collapsible arm 114 may comprise a first arcuate portion 181, a first substantially straight portion 183, a second arcuate portion 184, a third arcuate portion 186, and a second substantially straight portion 188. In the open position, the first arcuate portion 181 may be defined by a radius “r₂.” In the open position, the second arcuate portion 184 may be defined by a radius “r₃,” and the third arcuate portion 186 may be defined by a radius “r₄.” In the open position, the first substantially straight portion 183 may be formed in a straight section, an elliptical section, a circular section, or any other suitable shaped section. Additionally, in the open position, the second substantially straight portion 188 may be formed in a straight section, an elliptical section, a circular section, or any other suitable shaped section.

The arcuate portion 180 of the first collapsible arm 112 and the first arcuate portion 181 of the second collapsible arm 114 may be symmetrical, for example, r₁ may equal r₂. Additionally, the substantially straight portion 182 of the first collapsible arm 112 may be symmetrical to the first substantially straight portion 183 of the second collapsible arm 114. For example, the substantially straight portions 182, 183 may extend distally from the respective arcuate portions 180, 181 by a substantially identical distance. In various other embodiments, as shown in FIG. 19, the first collapsible arm and the second collapsible arm may be symmetrical or of any other shape that supports a specimen retrieval bag.

FIG. 5C illustrates a close-up view of the distal end of one embodiment of the specimen retrieval device 100 in the fired position. The rigidizable member 320 may connect a flexible portion 120 of the shaft assembly 106 to the first collapsible arm 112 and the second collapsible arm 114. The first collapsible arm 112 and the second collapsible arm 114 may be fastened to the rigidizable member 320 using any suitable fastening means, such as, welding, fusing, gluing, screwing, bolting, riveting, or any other suitable method. The rigidizable member 320 may be fastened to the flexible portion 120 of the shaft assembly 106 using any suitable fastening means, such as, welding, fusing, gluing, screwing, bolting, riveting, or any other suitable method.

In one embodiment, the first collapsible arm 112, the second collapsible arm 114, the knot pusher 118, and the rigidizable member 320 extend from a distal end 122 of the shaft assembly 106. The knot pusher 118 may be contained between the arm assembly 111 and the rigidizable member 320. In at least one embodiment, the collapsible arms 112, 114 may be formed of material that has a rectangular cross-section (i.e., substantially flat). In other embodiments, the collapsible arms 112, 114 may be formed of a material which has a circular cross-section, a square cross-section, or any other suitable cross-section.

FIG. 6 illustrates the proximal end of one embodiment of the specimen retrieval device 100 with the outer sheath 108 and the distal handle 104 removed. A substantial amount of force may be transmitted through the shaft assembly 106 during the action of translating the distal handle 104 proximally in the direction 109A (FIG. 5A) towards the proximal handle 102 to expose the specimen retrieval bag 110. As shown in FIG. 6, the shaft assembly 106 may comprise a hybrid shaft that further comprises the flexible portion 120 and a rigid portion 126. This combination of the flexible portion 120 and the rigid portion 126 may be required to overcome the substantial amount of force which may be transmitted through the shaft assembly 106 as the distal handle 104 is translated proximally.

In various embodiments, the rigid portion 126 may extend along a longitudinal axis “L” from the proximal handle to the flexible portion 120. The flexible portion 120 may extend along the longitudinal axis “L” from the rigid portion 126 to the distal end 122 of the shaft assembly 106. The flexible portion 120 may extend a distance which is greater than a distance extended by the rigid portion 126. For example, the rigid portion 126 may extend about 25 cm, whereas the flexible portion 120 may extend about 225 cm. In various embodiments, the flexible portion 120 and the rigid portion may be welded together or fastened using any suitable method for connecting the flexible portion 120 to the rigid portion 126. In at least one other embodiment, the flexible portion 120 and the rigid portion 126 may be formed of one piece of material. For example, the flexible portion 120 may be machined from the rigid portion 126. In various embodiments, the flexible portion 120 may be a flexible coil pipe, and the rigid portion may be a rigid shaft. During an operation, a surgeon may be able to deform the flexible portion 120 in any direction relative to the longitudinal axis “L” in order to assist the surgeon in placing the instrument where it is needed. For example, referring again to FIG. 1, the surgeon may manipulate the flexible portion 120 of the shaft assembly 106 in order to remove a gall bladder using the flexible endoscope 30. In order for the flexible portion 120 to move in a variety of directions during an operation, the outer sheath 108 may be fabricated of a flexible material to allow the flexible portion 120 to move in accordance with the surgeon's direction.

In various embodiments, in addition to the flexible portion 120, the specimen retrieval device 100 may allow for moving, angling, positioning, and placing of the arm assembly 111, and in particular for manipulating the specimen retrieval bag 110 relative to the shaft assembly 106. In certain embodiments, the arm assembly 111 can be rotated and/or translated relative to the shaft assembly 111, and/or the shaft assembly 106 can rotate and/or translate relative to the proximal handle 102. Manipulation, articulation, and rotation of the arm assembly 111 will allow the specimen retrieval bag 110 to be positioned at various locations during a surgical procedure, thereby providing the user with precise placement over the specimen retrieval bag 110 relative to an endoscope or a surgical target. A person skilled in the art will appreciate that the specimen retrieval device 100 has application in endoscopic procedures, laparoscopic procedures, and in conventional open surgical procedures, including robotic-assisted surgery.

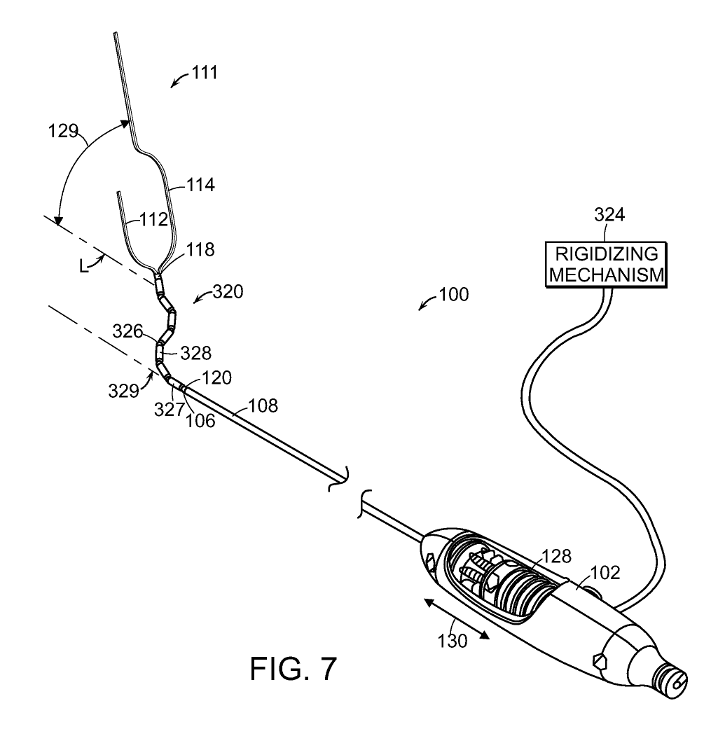

FIG. 7 illustrates one embodiment of the specimen retrieval device 100 shown in a manipulated position. FIG. 9 illustrates one embodiment of the specimen retrieval device 100 in yet another manipulated position. The manipulation may be possible through the use of the rigidizable member 320 and an external manipulator, for instance, a grasper inserted through a steerable, flexible endoscope; such manipulation is described in more detail herein. By rigidizable it is meant that the member 320 may be rendered substantially incapable of or resistant to bending and/or substantially incapable of compromise or flexibility. In the illustrated embodiment, the rigidizable member 320 is coupled to the rigidizing mechanism 324 via the proximal handle 102. The rigidizing mechanism 324 may be any device capable of rendering the rigidizable member 320 substantially rigid or inflexible. In various embodiments, the rigidizing mechanism 324 may be a wire tensioner, a vacuum pump, a combination of both, and/or other devices suitable to render the rigidizable member 320 substantially rigid upon actuation. The rigidizing mechanism 324 may be disposed within the proximal handle 102 or may be located remotely therefrom. The rigidizing mechanism 324 may be actuated or controlled by controls disposed on the proximal handle 102.

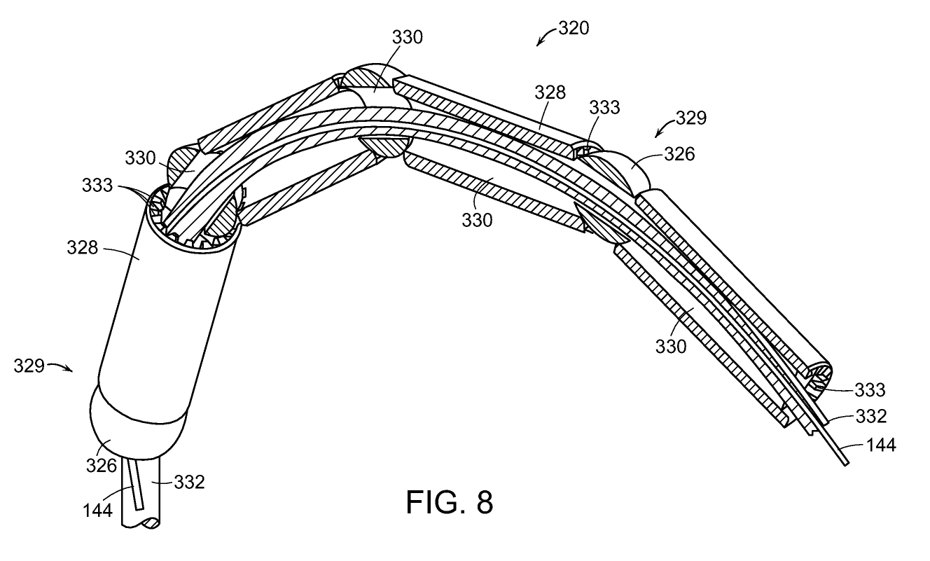

FIG. 8 illustrates a partial sectional view of one embodiment of the rigidizable member 320 in a manipulated position taken along the longitudinal axis with a tension wire extending through a central bore. The rigidizable member 320 also may comprise a central bore 330 defining a channel for receiving rigidizing components and end-effector control mechanisms, as needed. In the present embodiment, such an end-effector control mechanism is provided at least partially by a suture 144. The purpose of the suture 144 is to allow a surgeon or other operator of the rigidizable specimen retrieval device to close and/or cinch the specimen retrieval bag and is discussed in more detail herein.

A rigidizing component may be introduced in the central bore 330. A rigidizing component is any device or material suitable to render the rigidizable member 320 substantially rigid upon actuation of the rigidizing mechanism 324. In the rigid or inflexible mode, the rigidizable member 320 acts as a support for a manipulated, positioned, and angled end-effector, for instance, as previously discussed, a specimen retrieval bag and related components. Flexibility may be restored when the rigidizing component is deactuated or the rigidizing force is removed. This process may be repeated as necessary. In one embodiment, the rigidizing component may comprise one or more tensioning wires to apply a clamping force on the rigidizable member 320 to render them substantially rigid and inflexible. In another embodiment, discussed in more detail herein, the rigidizing component may comprise a state-change material disposed in the channel formed by the central bore 330 that becomes substantially rigid when a vacuum is applied to the rigidizable member 320. In various other embodiments, the rigidizing component may comprise a combination of tensioning wires and the state-change material and thus may employ a combination of tensioning force and vacuum to render the rigidizable member 320 substantially rigid. When the tensioning force or vacuum is released, the rigidizable member 320 return to their normally flexible state. In one embodiment, a flexible membrane may be provided over the rigidizable member 320. Among other functions, the flexible membrane may assist when a vacuum is applied to the rigidizable member 320 to actuate the state-change material. In other embodiments, the flexible membrane may function as a protective cover for the rigidizable member 320 when located inside a natural body orifice of the patient. Any of the tensioning components may be operated by the rigidizing mechanism 324, which is a general mechanism adapted and configured to apply a suitable force necessary to actuate the rigidizing components. The embodiments, however, should not be limited in this context.

Embodiments of the rigidizable member 320 may be formed in various shapes, sizes, and materials. In one embodiment, a rigidizable member may be formed with helical wires (e.g., coil spring). A flexible membrane may be provided over the rigidizable member 320. The rigidizable member 320 comprises a central bore that may be filled with biocompatible state-change material to render the rigidizable member 320 substantially rigid when a vacuum is applied. Alternatively, the rigidizable member may comprise a stiffening element configured to selectively stiffen when a vacuum force is applied thereto. The stiffening element may further comprise a flexible sheath and a plurality of elongate members disposed therein that are configured to generate friction therebetween. Such a stiffening element is described in more detail in commonly-owned U.S. application Ser. No. 11/952,475 to Stefanchik et al. and entitled SELECTIVE STIFFENING DEVICES AND METHODS, the disclosure of which is incorporated by reference in its entirety. In another embodiment, the rigidizable member 320 may be formed by connecting multiple cylindrical elements end-to-end held together by the flexible membrane. The cylindrical elements provide radial stiffness. The central bore or channel of the rigidizable member 320 may be filled with the biocompatible state-change material to render them substantially rigid when a vacuum is applied. A combination of tension wires may be added to provide additional rigidizing capability. Additional detail regarding rigidizable members may be found in commonly-owned U.S. application Ser. No. 11/707,831 to Stokes et al. and entitled RECONFIGURABLE ENDOSCOPE WITH LOCKING FEATURES, the disclosure of which is incorporated by reference in its entirety.

In the embodiments illustrated in FIGS. 7-9, the rigidizable member 320 may be formed with multiple assemblies 329 each comprising a ball 326 and a socket 328 and defining a central bore 330 (FIG. 8) therethrough. The ball 326 may be any spherical bead or element that may be insertable in a cylindrical sleeve such as the socket 326, such that in cooperation, the multiple ball 326 and socket 328 assemblies 329 render the rigidizable member 320 flexible in their normal state. The central bore 330 may be adapted to receive state-change material, one or more tension wires 332, or a combination thereof, to render the rigidizable member 320 substantially rigid whenever the rigidizing mechanism 324 is actuated by an operator or another device, for example. The ball 326 and socket 328 assemblies 329 may comprise a congruent pattern to provide additional locking force, and hence, additional rigidness.

In one embodiment, the rigidizable member 320 comprises a continuous length of assemblies 329 each comprising the nestable ball 326 and socket 328 components. In one embodiment, the ball 326 may be located (e.g., pressed) into and partially inserted into the socket 328 such that the ball 326 and socket 328 can rotate freely relative to each other and the ball 326 is retained within the socket 328. In one embodiment, the socket 328 may comprise projections 333 extending radially and inwardly and configured to engage and compress the surface of the ball 326. The ball 326 and the socket 328 components may be formed of stainless steel. In other embodiments, the ball 326 and/or the socket 328 may be formed of a suitable rigid biocompatible polymeric material or any combination of stainless steel and polymeric materials.

The nestable ball 326 and socket 328 components are disposed such that their adjacent surfaces coact. The adjacent ball 326 and socket 328 assemblies 329 are formed such that the ball 326 may be located (e.g., pressed) into the adjacent socket 328 and is retained therein. The projections 333 formed inside the socket 328 are adapted and configured to engage and compress the surface of the ball 326. The ball 326 and the socket 328 each have a central bore such that the multiple ball 326 and socket 328 assemblies 329 form the central bore 330 to accommodate the tension wire 332 extending therethrough. The tension wire 332 is fixedly attached to the distal end of the rigidizable member 320 and is coupled to the rigidizing mechanism 324 (FIG. 7) at the proximal end such that the tension wire 332 can be tensioned and/or relaxed. The tension wire 332 may be fixedly attached to the distal end of the rigidizable member 320 in any suitable manner such that the tension wire 332 is not pulled through the central bore 330 when the rigidizing mechanism 324 tensions the tension wires 332. For example, the tension wires 332 may comprise balls welded or molded onto the ends of the tension wires 332 and fixedly attached to the distal end of the rigidizable member 320 to ensure the tension wires 332 cannot be pulled through the central bore 330. Alternatively, terminations may comprise knots formed in the ends of the tension wires 332, or any suitable fastener or crimp may be provided to prevent the tension wires 332 from being drawn through the central bore 330 in operation. When the tension wire 332 is relaxed, the adjacent surfaces of the ball 326 and the socket 328 can rotate relative to each other and thus the rigidizable member 320 is rendered substantially flexible. In its normally flexible state, the rigidizable member 320 can move flexibly and slidably to conform to a manipulated position (FIG. 7). When the rigidizing mechanism 324 is actuated, the tension wire 332 imparts a load that clamps the adjacent surfaces of the ball 326 and socket 328 assemblies 329 together at its current relative orientation, thereby fixing or locking the shape of the rigidizable member 320. When the rigidizable member 320 is rendered substantially rigid, it may be used as a support for an end-effector, such as one comprising a specimen retrieval bag (FIG. 7). The tension wire 332 may be formed of any suitable material and in one embodiment may be formed of stainless steel.

In various embodiments, referring again to FIGS. 7 and 9, once the specimen retrieval bag 110 has been exposed, the operator of the specimen retrieval device 100 can manipulate a rigidizing knob 128 to actuate and deactuate the rigidizing component of the rigidizable member 320. While in a de-actuated state, the rigidizable member 320 is rendered substantially flexible and thus this may allow for easier manipulation and placement of the specimen retrieval bag 110 under the biological material to be removed. The manipulation may be performed on the arm assembly 111 and rigidizable member 320 by another device, such as a grasper fed through a working channel of an accompanying flexible endoscope. In addition, the knot pusher 118 may be manipulated in this manner. The rigidizing component, including here at least the tension wire 332 (FIG. 8), may be configured to connect to the rigidizing knob 128 to allow tension wire 332 to be set to a desired tension level and to be actuated and/or deactuated in conjunction with the movement of the rigidizing knob 128. The actuation/deactuation may be achieved by translating the rigidizing knob 128 proximally and/or distally within the proximal handle 102, as indicated by arrow 130 (FIG. 7). By translating the rigidizing knob, the tension wire 332 may be translated and locked proximally, thus locking the rigidizable member 320 in a substantially rigid and/or stiff configuration. The tension level may be altered and set by rotating rigidizing knob 129 in the direction indicated by arrow 132 (FIG. 9). By rotating the rigidizing knob 128, the amount of tension on the tension wire 332 may be adjusted by, for example, a drum assembly (not shown) around which the tension wire is wrapped internal to the proximal handle 102. In other words, rotating the rigidizing knob 128 may adjust the ultimate tension that may be provided by the tension wire 332 when the rigidizing knob 332 is translated proximally. Such adjustment permits varying amounts of rigidity and/or stiffness to be implemented in the rigidizable member 320 which may be advantageous where a completely rigid or stiff member is undesirable, for instance, where organ damage could occur from accidentally placing a rigidized end-effector in an inappropriate surgical location. The rigidizing force, here tension in the tension wire 332, may additionally be provided by the rigidizing mechanism 324 coupled to the rigidizable member 320 via the proximal handle 102 and the rigidizing knob 128 may further provide electrical signals to adjust and/or actuated/deactuate the rigidizing force provided by the rigidizing mechanism 324. The rigidizing mechanism 324 may, alternatively, be completely internal to the proximal handle 102.

For example, in FIG. 7, the arm assembly 111 has been manipulated from an initial position (e.g. FIGS. 5B-5C) and subsequently rigidized or locked at a position that is at least rotated in a direction indicated by first arrow 129. Similarly, in FIG. 9, the arm assembly 111 has been manipulated from an initial position (e.g. FIGS. 5B-5C) and subsequently rigidized or locked at another position that is at least rotated in a direction indicated by second arrow 131. Axes L′ and L″ are unique and parallel to the longitudinal axis L (FIG. 5B) of the shaft assembly 106. In addition to the several manipulated positions illustrated and described herein, the arm assembly 111, supported by the rigidizable member 320, may be translated and rotated to practically any desirable position and direction as appropriate and needed for a surgical task.

In various embodiments, the rigidizable member 320 can be coupled, including rotatably coupled, to the distal end of the shaft assembly 106. The illustrated embodiment includes a first socket 327 that is coupled to shaft assembly 106. Such coupling may use any suitable coupling means, such as, welding, fusing, gluing, screwing, bolting, riveting, or any other suitable method.

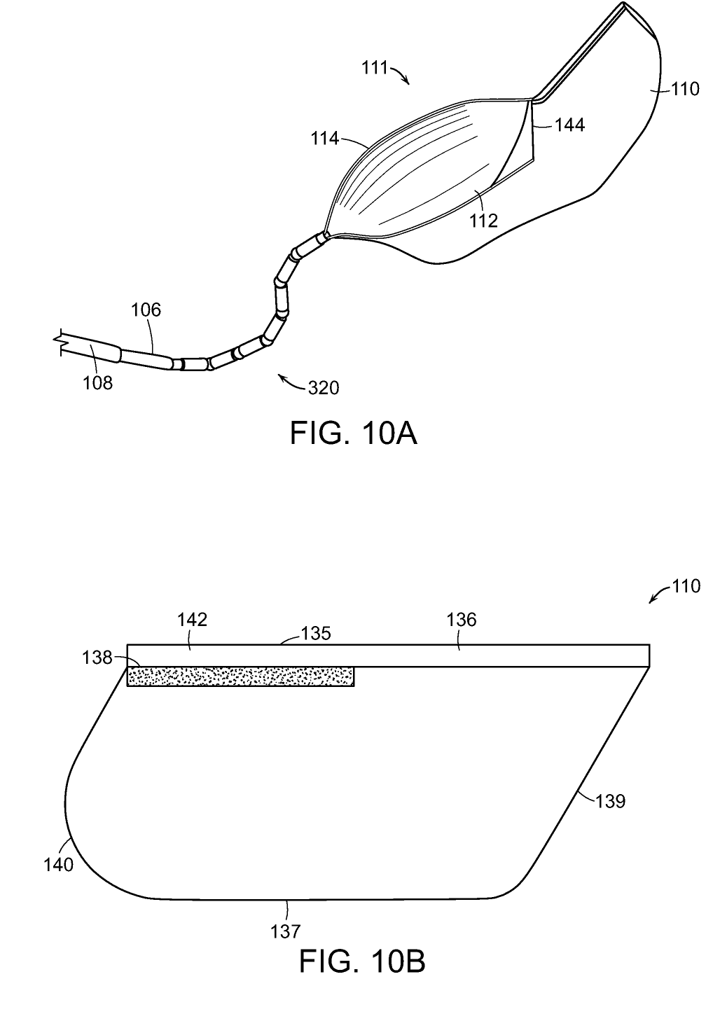

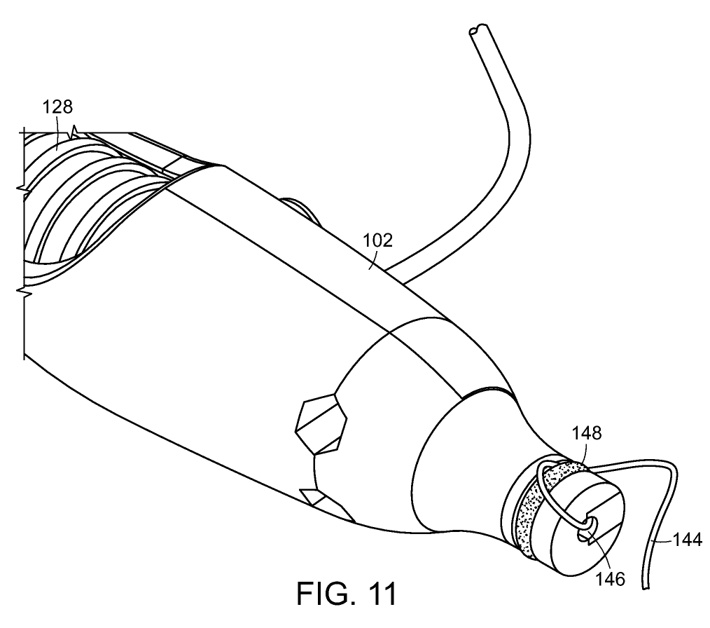

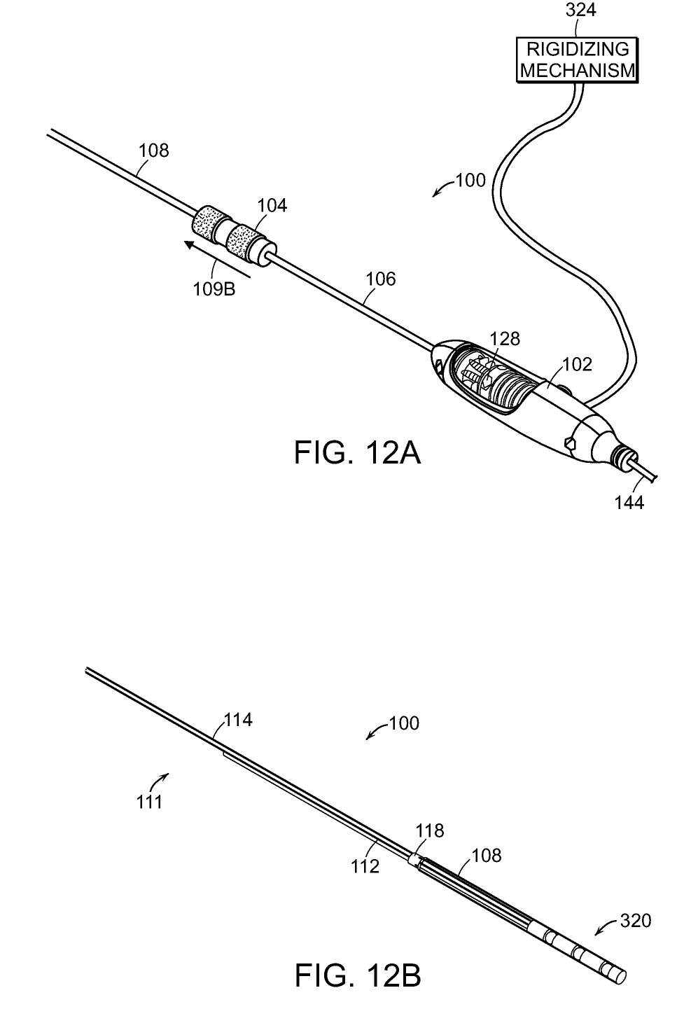

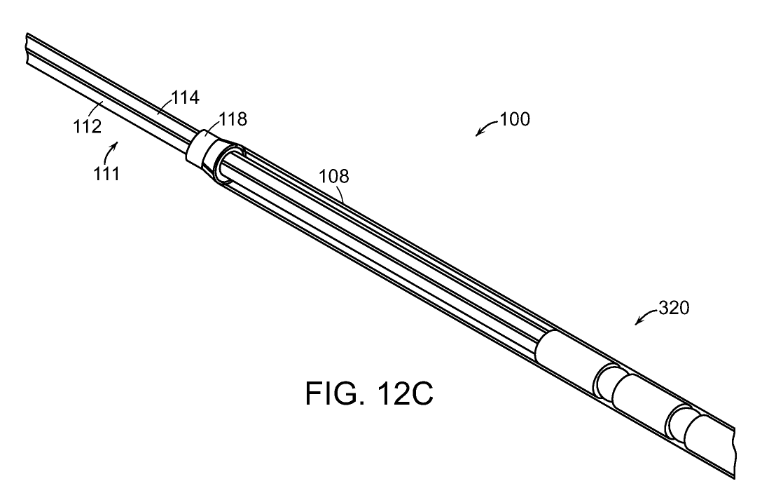

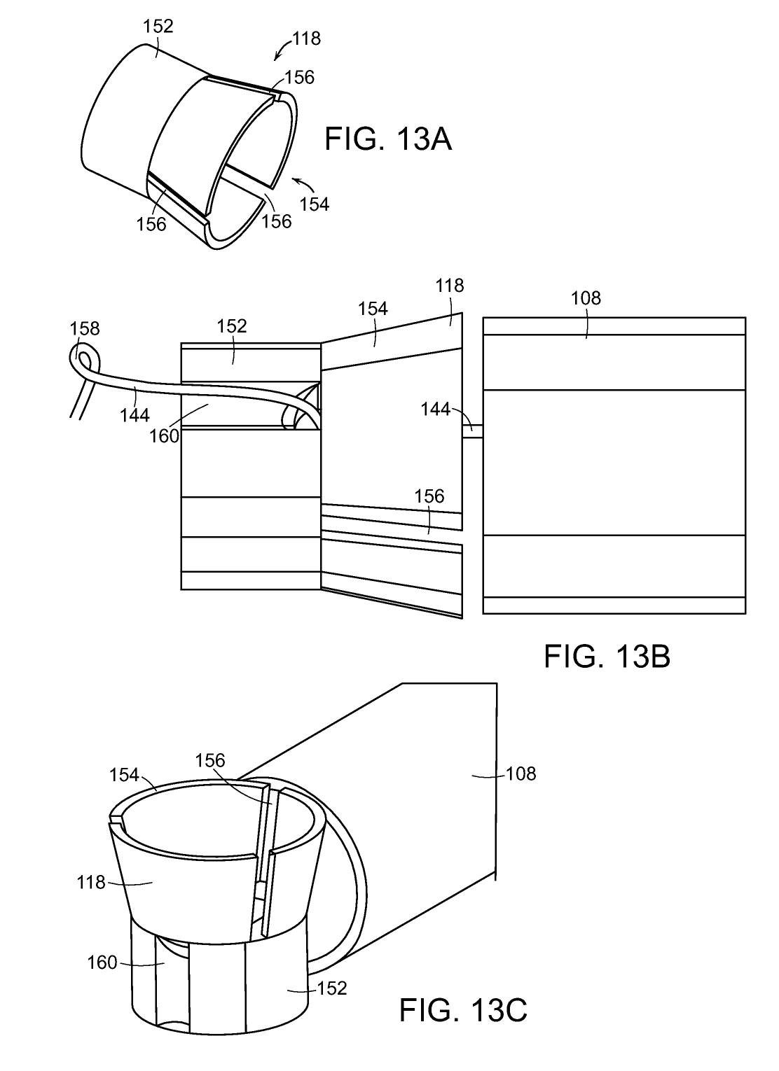

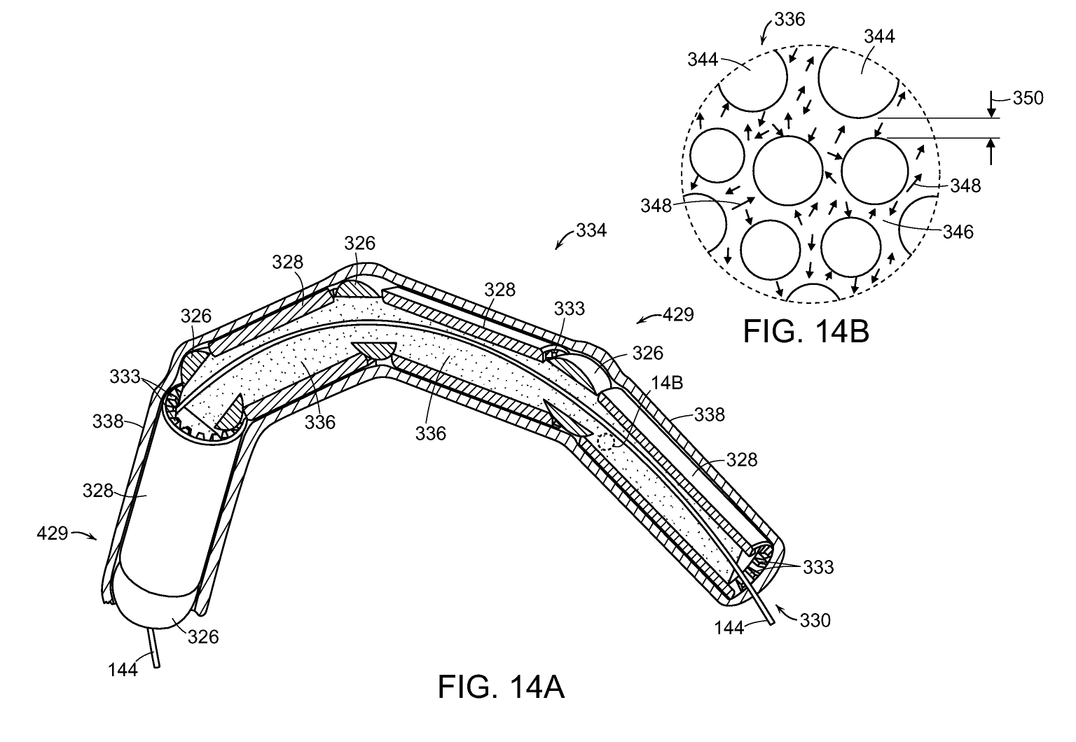

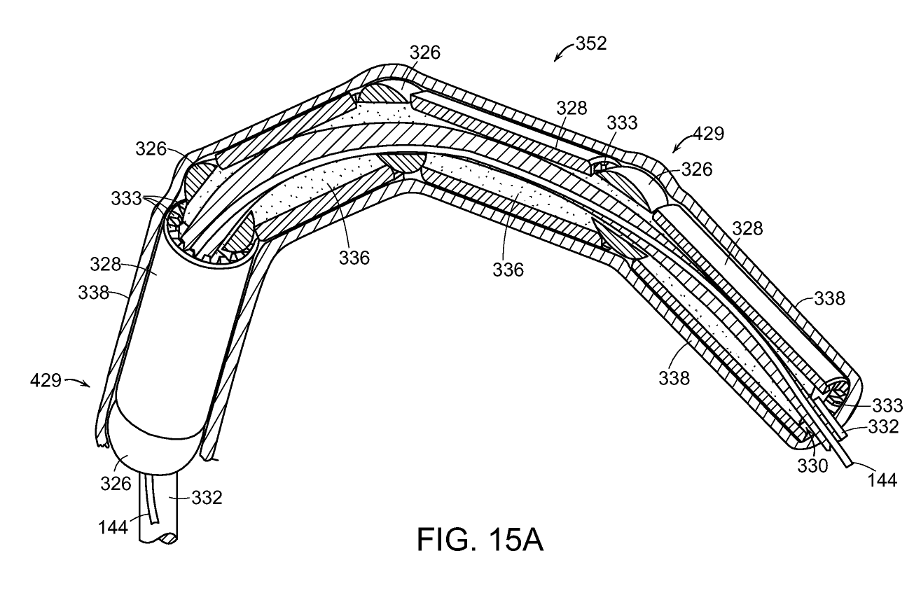

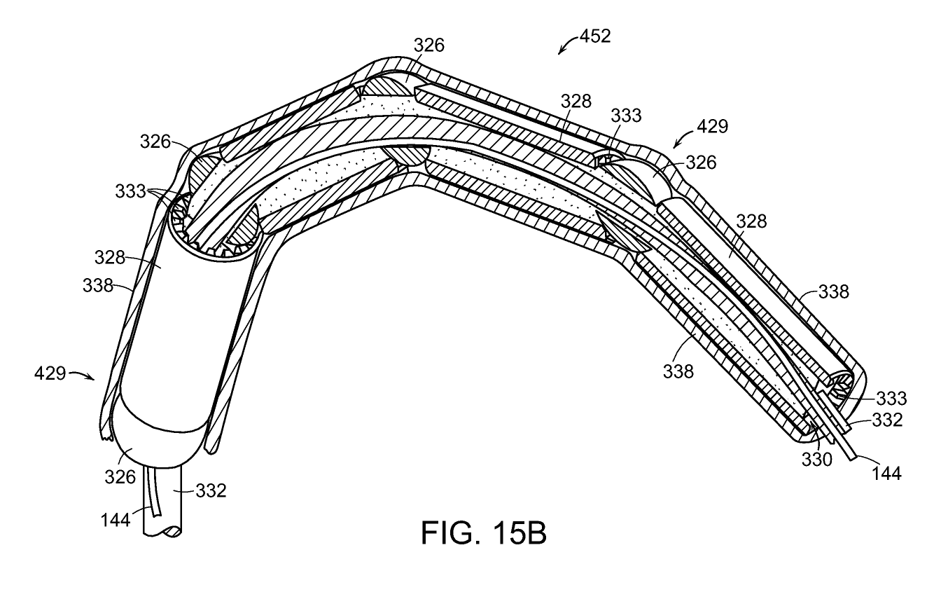

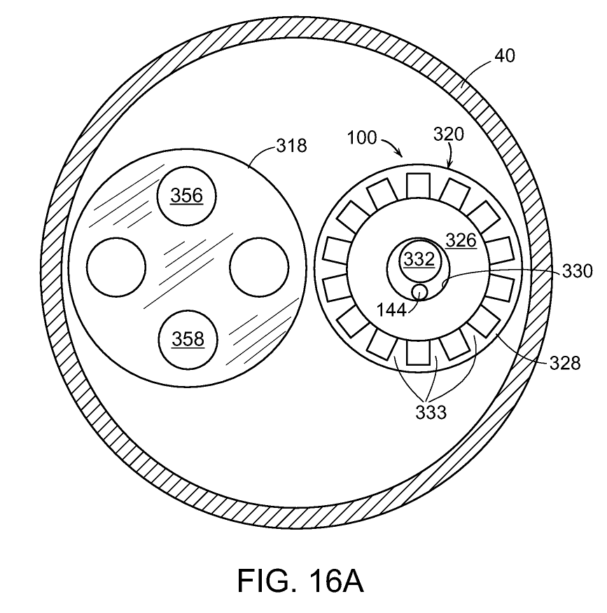

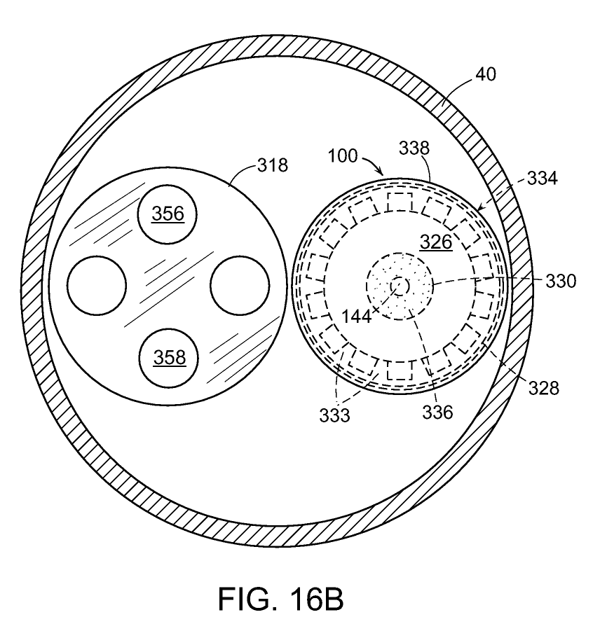

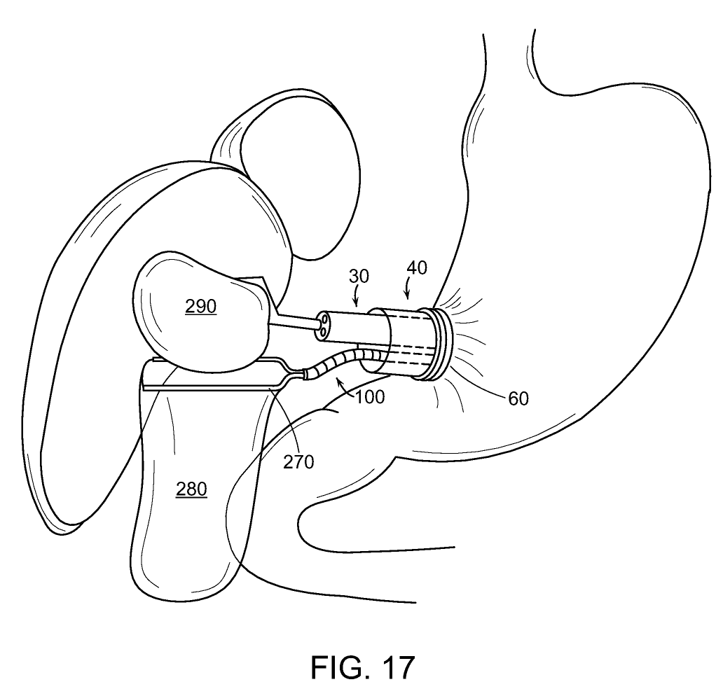

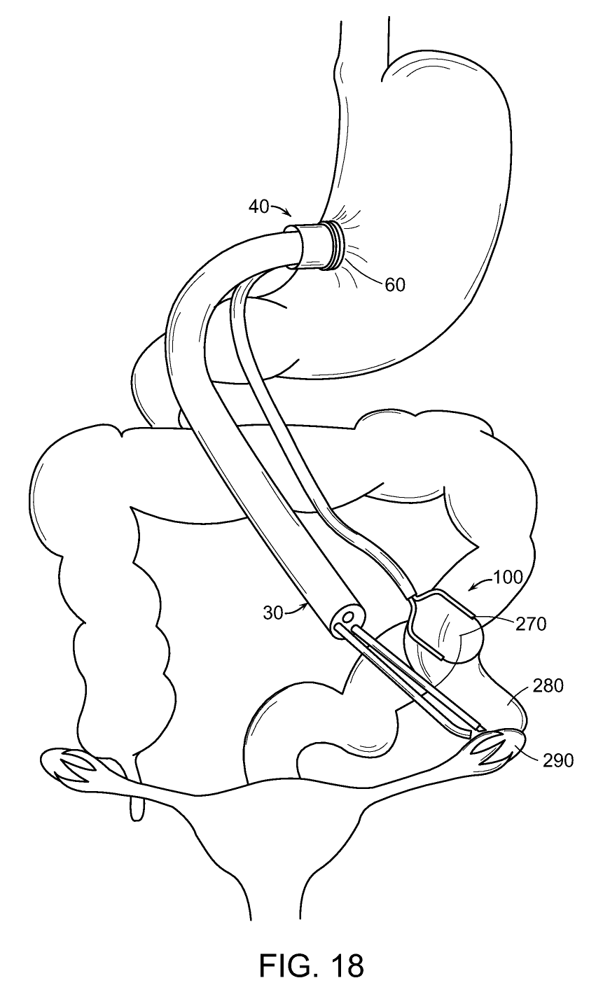

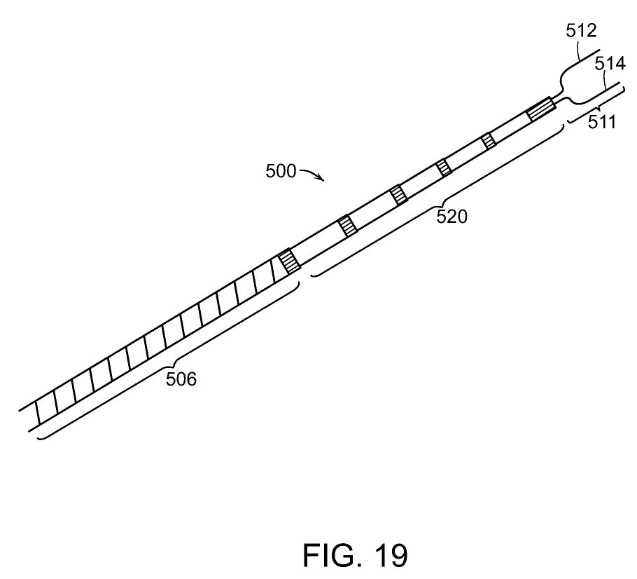

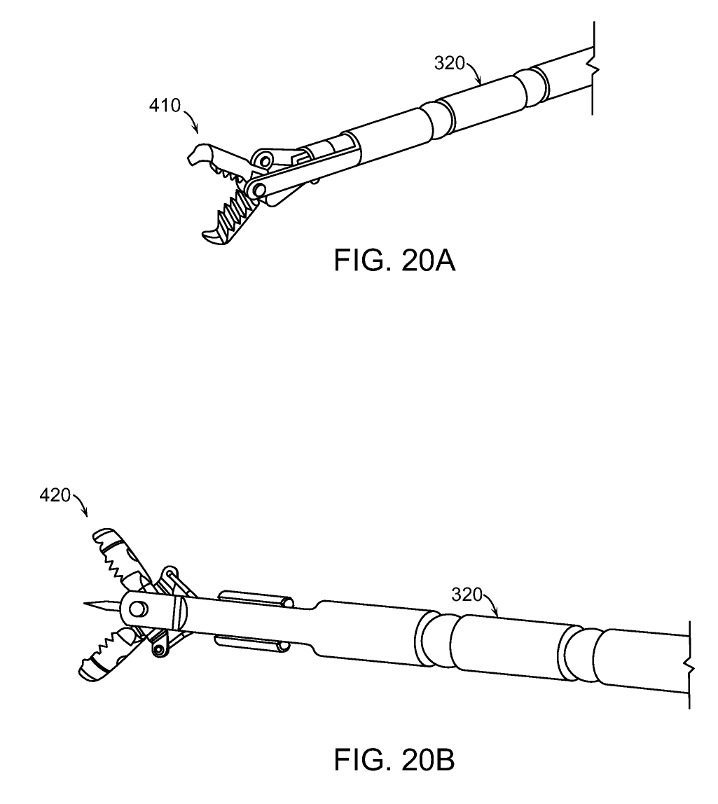

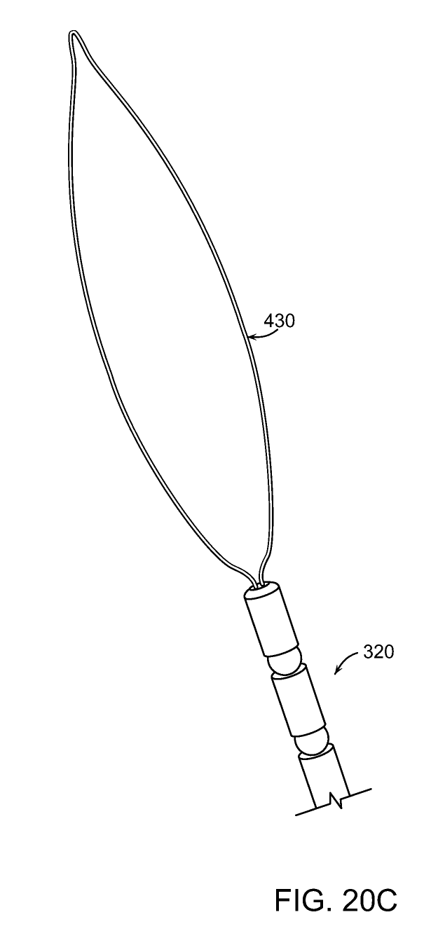

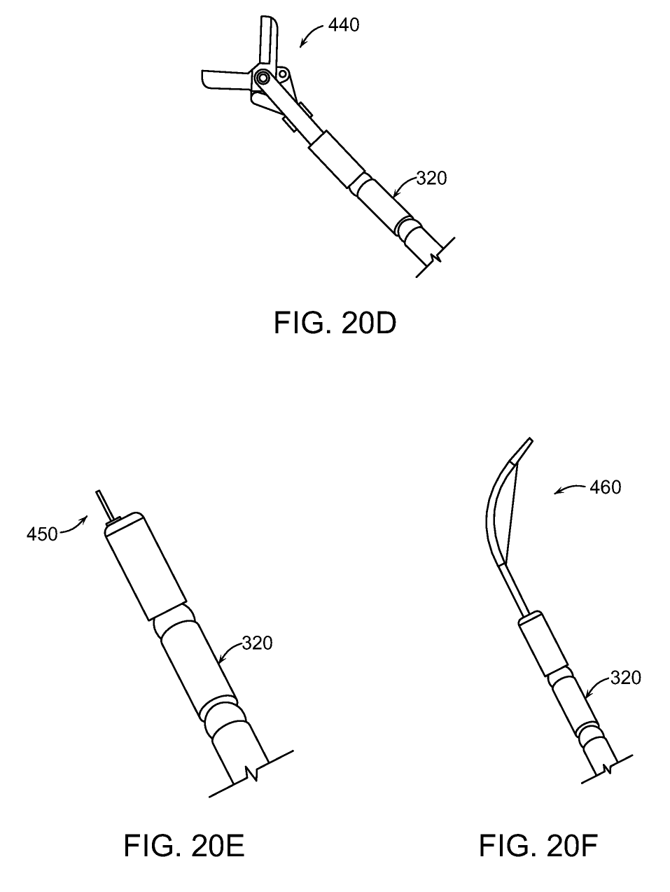

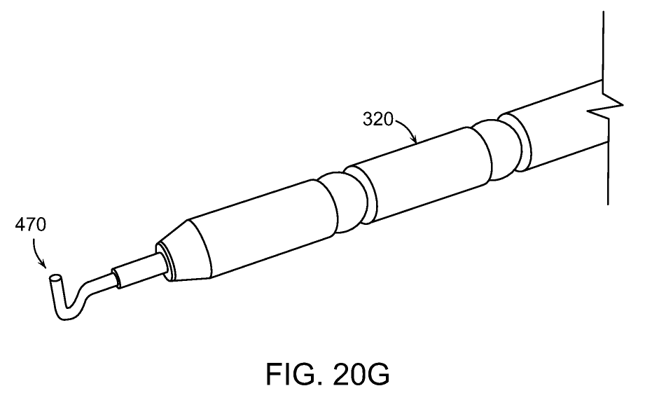

FIG. 10A illustrates one embodiment of a specimen retrieval bag 110 when the specimen retrieval device 100 is in the fired and yet another manipulated position. FIG. 10B illustrates a side view of one embodiment of the specimen retrieval bag 110. The specimen retrieval bag 110 may be configured to be retained on the arm assembly 111. In various embodiments, the specimen retrieval bag 110 may be rolled-up on the arm assembly 111 when the specimen retrieval bag 110 and arm assembly 111 are retained within the outer sheath 108 prior to firing of the specimen retrieval device 100. The manner in which the specimen retrieval bag 110 is rolled may be critical due to the operational environment of the specimen retrieval device 100. Given that the outer sheath 108 of the specimen retrieval device 100 may be passed through an overtube, or alternatively, the working channel of a flexible endoscope, the diameter of the outer sheath 108, and any item contained within the outer sheath 108, may be limited. For example, the outer sheath 108 may be required to fit in an overtube with an inner diameter of about 10-16 mm and typically about 13 mm and about 16 mm. Alternatively, the outer sheath 108 may be required to fit in a working channel with a diameter of about 2-5 mm and typically about 3.7 mm.

Although the diameter of the arm assembly 111 and the rolled-up specimen retrieval bag 110 may be limited due to the dimensional limits of the diameter of the outer sheath 108, a similar limit may not exist for the length of the arm assembly 111 and the specimen retrieval bag 110. For example, the length of the arm assembly 111 and the specimen retrieval bag 110 may be able to extend up to about 300 mm within the outer sheath 108. The relatively limited constraints on the length of the arm assembly 111 and the specimen retrieval bag 110 may be important to deliver a bag of significant volume to a surgical site. In at least one embodiment, the bag 110 may be rolled upon itself.

In the embodiment illustrated in FIG. 10B, the specimen retrieval bag 110 may comprise a top end 135 and a bottom end 137. The top end 135 may comprise an open portion 136 and a fused portion 138. The open portion 136 may be located near a proximal end 139, and the fused portion 138 may be located near a distal end 140. The fused portion 138 may be formed by fusing two portions of the specimen retrieval bag 110 together. The fused portion 138 may be formed by stitching, gluing, or using any other suitable method for forming a fused portion 138 of the specimen retrieval bag 110. The proximal end 139 may extend distally from the top end 135 to the bottom end 137, and the distal end 140 may extend distally from the top end 135 to the bottom end 137. The specimen retrieval bag 110 may be formed to allow the specimen retrieval bag 110 to be rolled up upon itself with a reduced diameter to meet the diameter requirements of the outer sheath 108 (FIG. 10A, for example).

With reference to FIGS. 10A and 10B, the first collapsible arm 112 may fit into a folded portion 142 on one side of the specimen retrieval bag 110 and the second collapsible arm 114 may fit into the folded portion 142 on the other side of the specimen retrieval bag 110. A suture 144 may run through the entire folded portion 142 and may be tied in a slip knot to allow the open portion 136 to be cinched once the biological material is put into the specimen retrieval bag 110. The asymmetric design of the arm assembly 111 enables the specimen retrieval bag 110 to receive biological material having a higher volume compared to symmetric designs. The first collapsible arm 112 and the second collapsible arm 114 may minimize buckling of the specimen retrieval bag 110 when the specimen retrieval bag 110 is in the rolled-up position.

FIG. 11 illustrates a close-up view of the proximal end of the proximal handle 102 of one embodiment of the specimen retrieval device 100. The suture 144 may extend through an opening 146 formed in the proximal end of the proximal handle 102. The suture 144 may terminate on the exterior of the proximal handle at an o-ring 148 or any other suitable assembly for retaining the suture 144. The specimen retrieval bag 110 may be removed from the arm assembly 111 once the biological specimen has been received in the specimen retrieval bag 110. First, the specimen retrieval bag 110 may be freed from the proximal handle 102 by pulling the suture 144 loose from the proximal handle 102. The suture 144 may extend from the folded portion 142 of the bag 110 at the distal end of the specimen retrieval device 100 through the center of the articulation actuator 170 and out of an opening 146 in the proximal end of the proximal handle 102.

FIG. 12A illustrates the proximal handle 102 of one embodiment of the specimen retrieval device 100 retracting the rigidizable member and the arm assembly 111 (FIG. 12B). Once the suture 144 has been freed from the proximal handle 102, the specimen retrieval bag 110 (FIGS. 10A, 10B) may be removed from the arm assembly 111. The specimen retrieval bag 110 may be removed from the arm assembly 111 by translating the distal handle 104 distally in the direction indicated by arrow 109B. As the distal handle 104 is translated distally, the outer sheath 108 moves distally to render the rigidizable member 320 co-axial in relation to the outer sheath 108 and to collapse the arm assembly 111 and receive the collapsed arm assembly 111 within the hollow lumen of the outer sheath 108. FIG. 12B illustrates one embodiment of the distal end of the specimen retrieval device 100 retracting the rigidizable member 320 and the first and second collapsible arms 112, 114 within the hollow lumen defined by the outer sheath 108. FIG. 12C illustrates a close-up view of one embodiment of the distal end of the specimen retrieval device 100 retracting the arm assembly 111. As the arm assembly 111 is retracted, the knot pusher 118 may be configured to be trapped at the distal end of the outer sheath 108 and remain trapped at the distal end of the outer sheath 108. As with FIG. 4B, the outer sheath 108 is shown transparent in FIGS. 12B-12C to illustrate internal components.

FIG. 13A illustrates one embodiment of a knot pusher 118. The knot pusher 118 may comprise a cylindrical portion 152 and a flared portion 154. In various other embodiments, the knot pusher 118 may not be limited to a cylindrical shape such as shown by the cylindrical portion 152 but may have a variety of configurations. In one embodiment, the knot pusher 118 may comprise an alternate distal portion, which may be formed in any suitable shape, such as a square or a rectangle, for example. Prior to firing the specimen retrieving device 100, the knot pusher 118 may be completely contained within the outer sheath 108 with the cylindrical portion 152 near the distal end of the outer sheath 108 and the flared portion 154 near the distal end of the rigidizable member 320. The flared portion 154 may be held within the outer sheath 108 in a substantially non-flared position as shown in FIG. 4B. This non-flared position may be attainable due to slots 156 located around the periphery of the flared portion 152. These slots 156 may be cut into the flared portion 152 to allow the flared portion 152 to be in a non-flared position when sufficient force is applied to the flared portion 152 and in a flared position (as shown in FIG. 13A) when a lack of sufficient force is applied to the flared portion 152. The knot pusher 118 may be fabricated of a resilient material, such as a resilient metal, plastic, or any other suitable material, to allow the flared portion 152 of the knot pusher 118 to expand to a flared position once the force is removed.

FIG. 13B illustrates one embodiment of the knot pusher 118 in the flared position interacting with the outer sheath 108 of the specimen retrieval device 108. As the outer sheath 108 is being retracted, the knot pusher 118 may eject from the exterior of the distal end of the outer sheath 108 and expand into the flared position. In one embodiment, the suture 144 may pass through the knot pusher 118 such that there may exist a knot 158 in the suture 144 at a distal end of the knot pusher 118. The suture 144 may enter the knot pusher 118 through an opening 160, which may comprise a slot, a hole, or any other suitable opening. In other embodiments, the opening 160 may be located in the cylindrical portion 152 of the knot pusher 118. The suture 144 may pass internally through the flared portion 152 and then exit the knot pusher 118 at the opening 160. The opening 160 may be configured to allow the suture 144 to pass through but not allow a knot 158 in the suture 144 to pass through the opening 160. The knot 158 may be formed at or near the distal end of the knot pusher 118 such that the knot 158 cannot be pulled proximally through the knot pusher 118.

FIG. 13C further illustrates one embodiment of the knot pusher 118 interacting with the outer sheath 108 of the specimen retrieval device 100. Once the specimen retrieval bag 110 has been removed from the arm assembly 111, the suture 144 may be pulled proximally from the proximal handle 102 (as shown in FIG. 11) to cinch the specimen device bag 110 closed. As the loose end of the suture 144 is pulled, the knot pusher 118 may rotate about an axis until the knot pusher 118 is engaged with the outer sheath 108 to further prevent the knot pusher 118 from entering the distal end of the outer sheath 108. In addition, the knot 158 of the suture 144 is pulled tight against the knot pusher 118. Once the knot 158 is secured against the knot pusher 118, the suture 144 may be pulled tight which may cinch the specimen retrieval bag 110. Once the bag 110 is cinched, elements of the specimen retrieval device 100, which may include the outer sheath 108, the arm assembly 111, the shaft assembly 106, and the rigidizable member 320, may be removed from the overtube 40, or alternatively the working channel 38 of the endoscope 30 (FIGS. 2A, 2B). This removal may occur to allow the operator, or surgeon, to place another instrument down the overtube 40 or the working channel 38 to complete the surgical procedure or perform another surgical procedure. Other elements of the specimen retrieval device 100, which may include the specimen retrieval bag 110, the suture 144, and the knot pusher 118, may be left in the patient until further procedures have taken place. The specimen retrieval bag 110 may remain at the distal end of the overtube 40, or alternatively, the flexible endoscope 30, with the suture 144 tethering the bag 110 to the proximal end where the operator may have control of the bag 110. Upon completion of the additional procedures, the specimen retrieval bag 110 may be extubated.

FIG. 14A illustrates a partial sectional view of one embodiment of a rigidizable member 334 taken along the longitudinal axis with a state-change material provided in a central bore. The rigidizable member 334 is similar to the rigidizable member 320 shown in the above illustrated embodiments, including that of FIG. 8. The rigidizable member 334 comprises a continuous length of assemblies 429 each comprising the coacting nestable ball 326 and socket 328 components with a state-change material 336 provided in the central bore 330. Additionally, suture 144 is provided in the central bore 330. The socket 328 comprises the projections 333 configured to engage and compress the surface of the ball 326. The state-change material 336 may be a biocompatible material suitable to render the rigidizable member 334 rigid when a vacuum is applied to the central bore 330 by the rigidizing mechanism 324 (e.g., a vacuum/pump arrangement in this embodiment) as shown in FIGS. 7 and 9, for example. To ensure an airtight seal between the coacting surfaces of the balls 326 and sockets 328 and to obtain suitable vacuum suction, a flexible membrane 338 is provided over the length of the rigidizable member 334. The flexible membrane 338 may be formed of any suitable flexible polymeric material, such as a suitable type of low stretch material like a polyester film, or a polymer film with some cord or fiber reinforcement.

In one embodiment, the state-change material 336 may comprise a material that behaves as a fluid and can take the shape or form of an object and when a vacuum is applied becomes solid and rigid. The state-change material 336 may be introduced into the central bore 330 as a fluid. The state-change material 336 fills the volume defined by the central bore 330 and conforms to its geometry. The state-change material 336 comprises hard solid bodies suspended in a liquid medium. A transition fluid creates a transition clearance between the hard solid bodies such that the state-change material 336 remains flexible. In this state, the adjacent surfaces of the balls 326 and the sockets 328 can rotate relative to each other and thus the rigidizable member 334 is rendered flexible and is able to flexibly move. A vacuum may be applied to the state-change material 336 to withdraw the transition fluid by suction. When the transition fluid is removed, the hard solid bodies contact each other and interlock the state-change material 336. The quantity of the transition fluid may be selected such that there is no appreciable change in volume when the transition fluid is removed. In the interlocked state, the hard solid bodies are packed together tightly to form a solid rigid component within the central bore 330 and thus fixes or locks the shape of the rigidizable member 334 rendering it rigid. When the rigidizable member 334 is rigid, it may provide support for a positioned end-effector such as one including a specimen retrieval bag. This process is completely reversible. Therefore, removing the vacuum and pumping the transition fluid back into the central bore 330 restores the clearance volume between the hard solid bodies to re-fluidize the rigid interlocked state-change material 336 and thus the rigidizable member 334 regains its flexibility.

FIG. 14B is an enlargement of one embodiment of a state-change material 336 that may be introduced into the central bore 330 for the purpose of rigidizing the rigidizable member 334. In the embodiment illustrated in FIG. 14B, the state-change material 336 is shown prior to a vacuum being applied to remove the transition fluid. In one embodiment, the state-change material 336 is a reversible state-changeable mixture comprising a plurality of hard solid bodies 344 and a carrier medium 346, with the carrier medium 346 filling any voids or interstices between the hard solid bodies 344. Within the mixture, the hard solid bodies 344 can be caused to transition from a formable state, preferably a near-liquid or fluent condition of mobility, to a stable, force-resisting condition through introduction and then extraction of a slight excess quantity of the carrier medium 346 beyond that required to fill the interstices of the hard solid bodies 344 when closely packed. In most embodiments, the carrier medium 346 is a liquid preferably excluding any air or other gases from the mixture. However, some embodiments may be use a carrier medium that is a liquid-gas froth. In one embodiment, the hard solid bodies 344 may be have a spherical form and may be surrounded by a liquid medium 346 with the same density as the bodies 344. The state-change material 336 also comprises an excess amount of liquid medium, hereinafter referred to as transition liquid 348. Pressure is applied against the hard solid bodies 346 to add a suitable quantity of the transition liquid 348 to create a small clearance volume 350. Otherwise, the hard solid bodies 344 are packed and nested against one another inside chamber the central bore 330. Therefore, the packed and abutted hard solid bodies 346 act as a solid fill in regard to their resistance to compression. The transition liquid 348 may be added to fill any added clearance volume. If the hard solid bodies 344 are of a small diameter, the added volume to allow clearance is also very small.

The state-change material 336 can be rapidly shifted from a formable (preferably near-liquid or fluent) state to a stable force-resisting state and back again to the formable state, through slightly altering the carrier-solid proportions of the state-change material 336 mixture. Embodiments are characterized by one or more of the following advantages: the ability to pressurize the state-change material 336 mixture and drive it against a surface as if it were a liquid; the ability to conform due to the negligible volumetric change that accompanies a state change; the ability to effect the state-change with a very small volume of single-constituent transfer and with consequently small actuation devices without the need for a vacuum pump, without chemical reactions, and with no need for thermal or electrical energy to be applied to the mixture; and the ability to tailor the mixture to satisfy a wide variety of physical specifications in either the flowable or the rigid stable state.

The state-change material 336 mixture can be used to fill the volume defined by the central bore 330 and is reusable. The state-change material 336 mixture can also be used in any product or shape that benefits from the incorporation of arbitrary reformability or precise reconfigurability. The state-change material 336 mixture provides useful properties for use in a supportive element or apparatus such as the rigidizable member 334.

The state-change material 336 mixture in its formable state may be loosely compared to quicksand, while the state-change material 336 mixture in its stable state may resemble hard-packed sand or even cement, with the transition being caused by the transfer of a relatively small amount of liquid. Hence the state-change material 336 mixture, while in the formable state, includes enough liquid 346 to fill the interstices between the nested solid bodies 344, and an excess amount of liquid that is referred to as the transition liquid 348. In the stable state the transition liquid 348 is absent and the hard solid bodies 344 are completely packed or nested.

In one embodiment, the hard solid bodies 344 are uniform, generally ordered, and closely spaced, with the predominate mass of the hard solid bodies 344 closely-packed and touching. To create mobility, the transition liquid 348 is introduced in just-sufficient quantity to create a fluent condition by providing the clearance 350 between some of the hard solid bodies 344, which clearance permits the introduction of at least two simultaneous slip planes between ordered masses of the hard solid bodies 344 at any point in the state-change material 336 mixture. The hard solid bodies 344 themselves separate freely from one another under movement of the liquid and without turbulent mixing, and shift relative to one another generally in ordered bulk masses. The hard solid bodies 344 should be of a density that is close enough to that of the liquid 346 to permit flow of the hard solid bodies 344 along with the liquid 346, or should have a size or structure that facilitates movement of the hard solid bodies 344 along with the liquid 346.

In a method according to one embodiment, the state-change material 336 mixture while in the formable state is first made to conform to the volume define by the central bore 330. The hard solid bodies 344 in the state-change material 336 mixture are then caused to transition from the fluent condition to the stable condition through extraction of the transition liquid 348. This extraction removes the clearance volume 350 required to provide slip-planes between ordered masses of the hard solid bodies 344, thereby causing the hard solid bodies 344 to make nested, packed, interlocking or otherwise stable consolidated contact. The state-change material 336 mixture, now in the stable state, has a surface that conforms to the central bore 330.

Distribution of uniform pressure against the surface of each hard solid body 344, coupled with the clearance volume 350 furnished by the transition liquid 348, assures that the hard solid bodies 344 are not forced against one another while the mixture is in the fluent condition. This elimination of body-to-body compression forces in turn prevents the hard solid bodies 344 from sticking together and resisting displacement while the mixture is in the fluent condition. Pressure forces in the liquid 346 may be induced by a two-way pump or other transfer system.

The hard solid bodies 344 themselves may have various geometries and may be provided within the state-change material 336 mixture in one uniform type, or there may be two or more types or sizes of bodies dispersed or layered within a mixture. For example, spherical bodies of one size might have smaller bodies filling the interstices between the larger bodies, or a layer of short fiber bodies might float above a layer of spherical bodies. Flake-like bodies also can be used, in which case the flat faces of the bodies can be pressed against one another to create a force-resisting body mass. The flat faces provide many times the contact area of abutting spheres, with accordingly higher friction or adhesion potential when consolidated against one another. If the flakes are in the form of a laminate that has one side heavier than the carrier medium and one side lighter, and if the flakes are closely spaced and in a medium which suppresses turbulence and solid body tumbling, the bodies will tend to be supported in, and to be consolidated in, an ordered parallel configuration. In this case, as with the spherical bodies, the transition liquid quantity will be just sufficient to create shear motion of body masses under low displacement forces. State-change material 336 mixtures with more than one type or size of body can be used with the bodies either intermingled or separately layered, as by differing densities or the inability of bodies of one layer to pass through bodies in an adjacent layer. Bodies of different sizes or types also may be separated from one another by flexible or extensible porous materials or fabrications that allow passage of liquids but not the confined bodies. The degree of accuracy or irregularity on the surface of a stabilized mass of the mixture may depend upon the relationship between the fineness of the bodies and the dimensions to be captured, and the size and degree of regular packing order of the solid bodies. If the bodies are very small compared to the contours of a shape that is to be replicated, or if the interstices between larger bodies in the mixture are filled by such smaller bodies, the mobile solid bodies of the mixture will consolidate and assume a near-net shape relative to any impressed shape when the transition liquid is extracted from the mixture. A more detailed description of the state-change material 336 is provided in U.S. Pat. No. 7,172,714 to Jacobson, and U.S. Pat. No. 6,780,352 to Jacobson, which are both incorporated herein by reference.

In another embodiment (not illustrated), in place of or in addition to the state-change material, a plurality of elongate members may extend through the rigidizable member. Any number of elongate members can be used, and the number selected will be dependent on a variety of factors, such as the material used, the shape and size of each elongate member, and the flexibility or stiffness of each elongate member. Furthermore, the elongate members can have any size and shape that allows them to be disposed, at least partially, in the flexible membrane. In one embodiment the elongate members are flexible, thin, and non-elastic. The elongate members can be made of a material with a high coefficient of friction, for example, a coefficient of friction in the range of about 0.8 to about 2. In a preferred embodiment, the coefficient of friction is about 2. For example, the elongate members can be made of steel. Although the thickness of the elongate members can vary, in an exemplary embodiment the elongate members have a thickness of about 0.5 mm. The elongate members also have a length that can vary depending on the desired use. The length of the elongate members can be approximately shorter, longer, or the same size as the length of the rigidizable member. The length of the elongate members may be slightly less than the length of the rigidizable member. In other embodiments the length of the elongate members can be approximately half as long, or less than half as long, as the length of the rigidizable member. Further, each of the elongate members may have dissimilar lengths. The elongate members can taper at their respective ends, much the same way bristles on a paint brush taper, or they can have a random assortment of lengths.

The elongate members can have a variety of different configurations that allow them to be stiffened. In one embodiment of a rigidizable member, or stiffening element, the elongate members are in the form of circular wires. In another embodiment of a rigidizable member, the elongate members are in the form of planar strips. The planar strips in the rigidizable member can allow for bending in only a single plane. This can be accomplished, for example, by forming the strips such that a height of the strip is less than a width of the strip. Accordingly, bending can occur in a first direction, i.e. along the width, while bending can be prevented in a second direction, i.e. along the height.

The elongate members can be configured to generate friction therebetween. There are many ways by which the elongate members can generate friction. In one embodiment the elongate members can include surface features that increase the friction between adjacent members. For example, surfaces of the elongate members can be made rough, for example, by sand-blasting the surfaces. Other techniques for making a surface rough or cratered can be used. The surfaces of each of the elongate members can then bind or grip against each other when a vacuum force is applied. The surface features can be such that the elongate members can continue gripping each other even after the outside force is no longer applied, or alternatively, such that the gripping ceases when the outside force is no longer applied.

The elongate members can be arranged within the rigidizable member in a variety of ways. In one embodiment, the elongate members can be located at a distal end of the rigidizable member. The elongate members can be configured to be anchored to a portion of the flexible membrane, including an end of the flexible membrane, or alternatively, they can remain free. Further, the elongate members can substantially fill a volume of the rigidizable member. However, it is preferable to have some space between the elongate members and/or between the elongate members and the flexible membrane to allow the elongate members to slidably move and flex and to be engaged by the rigidizable member when a vacuum force is applied thereto. In one embodiment the elongate members are arranged in one or more bundles. A more detailed description of the elongate members is provided in commonly-owned U.S. application Ser. No. 11/952,475 to Stefanchik et al. and entitled SELECTIVE STIFFENING DEVICES AND METHODS, the disclosure of which is incorporated by reference in its entirety.

FIG. 15A illustrates a partial sectional view of one embodiment of a rigidizable member 352 taken along the longitudinal axis. The rigidizable member 352 is similar to the rigidizable member 320 and 334 shown in the above illustrated embodiments, including that of FIGS. 8 and 14A. The rigidizable member 352 comprises a continuous length of assemblies 429 each comprising the coacting nestable ball 326 and socket 328 components. The socket 328 comprises the projections 333 configured to engage and compress the surface of the ball 326. A combination of the tension wire 332 and the state-change material 336 are provided in the central bore 330. Additionally, the suture 144 is provided in the central bore 330. The flexible membrane 338 is provided over the length of the rigidizable member 352. The flexible membrane 338 may be formed of any suitable material as previously described. In the ball 326 and socket 328 assembly 429, the socket 328 is substantially smooth.

A vacuum generated by a portion of the rigidizing mechanism 324 (FIGS. 7, 9) may be applied to the central bore 330 via vacuum ports (not shown) at the proximal end of the rigidizable member 352 to remove the transition fluid 348 in the central bore 330 and cause the hard solid bodies 344 to be nested, packed, interlocked or otherwise coupled in substantially rigidly stable consolidated contact. Thus, the state-change material 336 transitions state from a fluent state to a solid rigid state to fix and lock-in the shape of the rigidizable member 352 rendering it rigid. If additional rigidity is required, tension may be applied to the ball 326 and socket 328 assemblies 429 by tensioning the tension wire 332 with a wire tensioner portion of the rigidizing mechanism 324. Thus, the rigidizable member 352 may be first manipulated by, for example, an endoscope, and then rendered substantially rigid to provide a support for an end-effector inside a patient. Because, the process is completely reversible, removing the tension on the tension wire 332 and pumping the transition fluid 348 back into the central bore 330 re-fluidizes the packed interlocked hard solid bodies 344 (FIG. 14B) of the state-change material 336 and the rigidizable member 352 regains its flexibility. In its normally flexible state, the rigidizable member 352 may be manipulated inside the patient.

FIG. 15B illustrates a partial sectional view of one embodiment of a rigidizable member 452 taken along the longitudinal axis. The rigidizable member 452 is similar to the rigidizable members 320, 334, and 352 shown in the above illustrated embodiments, including that of FIGS. 8 and 14A, and 15A. The rigidizable member 452 comprises a continuous length of assemblies 429 each comprising the coacting nestable ball 326 and socket 328 components. The socket 328 comprises the projections 333 configured to engage and compress the surface of the ball 326. The tension wire 332 and suture 144 are provided through the central bore 330. In the embodiment illustrated in FIG. 15B, no state-change material is provided in the central bore 330. The flexible membrane 338, however, is provided over the length of the rigidizable member 334. The flexible membrane 338 may be formed of any suitable material as previously discussed. In the ball 326 and socket 328 assembly 429, the socket 328 is substantially smooth.

FIG. 16A is a cross-sectional view of one embodiment of a system including an endoscope inserted through an overtube and a portion of a rigidizable surgical instrument also inserted through the overtube and adjacent to the endoscope. Shown within the overtube 40 are an endoscope 318 and a rigidizable member 320. The rigidizable member 320 is similar to the rigidizable member 320 shown in FIG. 8. The rigidizable member 320 may be positioned of and moved independently of the endoscope 318 along a longitudinal axis of the hollow overtube 40. The end portion of the socket 328 also may comprise the projections 333 configured to engage and compress the ball 326 component. The endoscope 318 may have an adjustable portion (i.e., the flexible, steerable articulating section) that is usually the distal 12 cm to 16 cm portion of the endoscope 318. The endoscope 318 comprises a viewing element 356 and one or more working channels 358. The endoscope 318 may be steered using two or more wires using generally well known techniques.

The rigidizable member 320 also comprises the central bore 330 defining a channel. The tension wire 332 and the suture 144 are disposed in the central bore 330. The tension wire 332 is employed to render the rigidizable member 320 rigid and prevent it from flexing or bending upon the application of a rigidizing force. The tension wire 332 is fixedly attached to the distal end of the rigidizable member 320 in any suitable manner such that the tension wire 332 is not pulled through the central bore 330 when tensioning the tension wires 332 as previously discussed. The tension wire 332 may be operated such that the rigidizable member 320 may be in a rigid state. Flexibility is restored when the tensioning force is removed. The process may be repeated as necessary. In one embodiment, when activated, the tension wire 332 applies a clamping force on the rigidizable member 320 to render it rigid or firm and difficult to bend or flex. When the tensioning force is released, the rigidizable member 320 returns to its normally flexible state. The tension wire 332 may be actuated by a wire tensioner or other rigidizing mechanism 324 (FIGS. 7, 9).

Embodiments of the rigidizable member 320 may be formed in various shapes, sizes, and materials. In one embodiment, the rigidizable member may be formed with helical wires (e.g., coil spring). A highly flexible sheath may be provided over the rigidizable member 320. A central bore 330 through the rigidizable member 320 may be filled with biocompatible state-change material 336 or elongate members to render the rigidizable member rigid or stiff when a vacuum is applied to the central bore 330. In another embodiment, rigidizable members may be formed by connecting multiple cylindrical elements held together with a highly flexible sheath. The cylindrical elements provide radial stiffness. The central bore 330 may be filled with a combination of the state-change material 336 and the rigidizing may be assisted by employing the one or more tension wires 332.

FIG. 16B is a cross-sectional view of another embodiment of a system including an endoscope inserted through an overtube and a portion of a rigidizable surgical instrument also inserted through the overtube and adjacent to the endoscope. Shown within the substantially hollow overtube 40 is the endoscope 318 and a rigidizable member 334 covered with a first flexible membrane 338. The rigidizable member 334 may be positioned and moved independently of the endoscope 318 along a longitudinal axis of the hollow overtube 40. The rigidizable member 334 is similar to the rigidizable member 334 shown in FIG. 14A. The state-change material 336 is provided in the central bore 330. The flexible membrane 338 is similar to the flexible membrane shown in FIG. 14A. To ensure an airtight seal between the coacting surfaces of the ball 326 (FIG. 14A, for example) and socket 328 assemblies and to obtain suitable vacuum suction, the flexible membrane 338 is provided over the length of and over the distal end of the rigidizable member 334. As previously discussed, the flexible membrane 338 may be formed of any suitable flexible polymeric material, such as a suitable type of low stretch material like a polyester film, or a polymer film with some cord or fiber reinforcement. The end socket 328 also may comprise the projections 333 configured to engage the surface of the ball 326.

With reference now to FIG. 17 and FIGS. 1, 2B-5C, 7, 9, and 13B, FIG. 17 illustrates one method of employing a rigidizable specimen retrieval device and an endoscope through an overtube to perform a cholecystectomy. Here, an overtube 40 has been advanced through an incision, an otomy site 60, in the stomach, similar to that shown in FIG. 1. First, a flexible endoscope 30 and a rigidizable specimen retrieval device 100 are advanced independently into a patient through an overtube, or other suitable access device. Then the specimen retrieval device 100 is fired, opening the collapsible arms 270 and specimen retrieval bag 280, as previously discussed with reference to FIGS. 3A-5C. A grasper, or other appropriate endoscopic tool, is inserted through a working channel 38 (FIG. 2B) of the endoscope 30 and then used in conjunction with the steerable section of the flexible endoscope 30 to manipulate and position the collapsible arms 270 and the specimen retrieval bag 280. After proper positioning, the rigidizable member 320 (FIG. 7) is rendered rigid by actuation of the rigidizing mechanism 324 (FIGS. 7, 9). Subsequently, an appropriate cutting tool is inserted into one of the working channels 38 of the endoscope 30 and is used to cut a surgical target 290. The surgical target 290 is then placed into the specimen retrieval bag 280 and the suture 144 is loosened from the proximal handle 102. The rigidizable member is then rendered flexible by deactuation of the rigidizing mechanism 324. The outer sheath 108 (FIGS. 3A-5C) is next advanced back over the rigidizable member 320 to render it approximately co-axial with the outer sheath 108, similar to its pre-fired position. Distal movement of the outer sheath 108 also advances the knot 158 (FIG. 13B) and re-collapses the collapsible arms 270 as previously discussed. The specimen retrieval bag 280, now loosened from the collapsible arms 270, is cinched by pulling on the suture 144, thus providing sterile capture of the surgical target 290. Removal of the specimen retrieval bag 280 can now be performed when the procedure is completed by extubating it back through the overtube 40.

With reference now to FIG. 18 and FIGS. 2B, 7, and 17, FIG. 18 illustrates another method of employing a rigidizable specimen retrieval device and an endoscope through an overtube to perform a oophorectomy. The procedure for this embodiment is similar to that previously described with reference to FIG. 17. In both embodiments, the rigidizable member 320 (FIG. 7) allows positioning of the specimen retrieval bag 280 or other end-effector. This enhanced positioning of an end-effector may be especially helpful when the surgical target 290 is not in close proximity to the otomy site 60, as shown in FIG. 18. Furthermore, one embodiment of a rigidizable surgical instrument may be passed outside the endoscope 30 and not inserted into any of the working channels 38 (FIG. 2B) of the endoscope 30. This keeps the working channels 38 of the endoscope 30 open for insertion of additional surgical tools.

When called for above, and in various embodiments, tension is applied to the tension wire 332 by the rigidizing mechanism 324 located either outside or within the proximal handle 102 (FIGS. 7 and 9). This renders the rigidizable member 320 (FIGS. 7 and 9) rigid. When flexibility of the rigidizable member 320 is necessary, tension is removed from the tension wire 332 by deactuation of the rigidizing mechanism 324.

Also, with reference now to FIGS. 14A and 14B, a procedure similar to that previously described with reference to FIGS. 17 and 18 may be employed for the rigidizable member 334 comprising the state-change material 336 or elongate members in the central bore 330. In such embodiments, a vacuum and pump mechanism may be employed to rigidize and restore flexibility to the rigidizable member 334. For example, as previously discussed, a vacuum may be applied to the central bore 330 to withdraw the transition liquid 348 to render the rigidizable member 334 rigid. A pump may then be employed to pump the transition liquid 348 back into the central bore 330 to restore the flexibility to the rigidizable member 334. Likewise similar procedures may be applied to the rigidizable member 354.

FIG. 19 illustrates one embodiment of a rigidizable specimen retrieval device 500. Here, the specimen retrieval device comprises a specimen retrieval section 511, a rigidizable member 520 and a coil pipe 506. The specimen retrieval section 511 comprises collapsible arms 512, 514 which are symmetrical. The remaining components may be similar to those of the embodiments described above.

The embodiments described with reference to FIGS. 1-19 provide, among other things, the specimen retrieval bag 110 and related components as an end-effector. Various end-effectors may be substituted for the specimen retrieval bag 110 and are within the scope of the appended claims. FIGS. 20A-G illustrate various embodiments of a rigidizable surgical instrument employing various embodiments of end-effectors. For example, the various embodiments of the end-effectors include, but are not limited to, grasper jaws 410 (FIG. 20A), biopsy jaws and a spike 420 (FIG. 20B), a snare loop 430 (FIG. 20C), scissors 440 (FIG. 20D), a needle knife 450 (FIG. 20E), sphincterotome 460 (FIG. 20F), and a hook knife 470 (FIG. 20G). The above end-effectors are described in more detail in commonly-owned U.S. application Ser. Nos. 11/610,803 and 12/133,953 both to Nobis et al. and both entitled MANUALLY ARTICULATING DEVICES, the disclosures of which are incorporated by reference in their entirety.

Additional figures are provided to illustrate some, but not all, end-effectors that may be implemented with a rigidizable member 320 according to the present invention. The rigidizable member 320 is similar to that described above. The respective end-effector may be connected to the rigidizable member 320 through any suitable fastening means which may include fusing, welding, gluing, bolting, riveting and/or screwing, for example. FIG. 20A is a perspective view of one embodiment of an end-effector having grasper jaws 410 for use with a rigidizable member 320. FIG. 20B is a perspective view of one embodiment of an end-effector having opposed biopsy jaws and a spike 420 for use with a rigidizable member 320. FIG. 20C is a perspective view of one embodiment of an end-effector having a snare loop 430 for use with a rigidizable member 320. FIG. 20D is a perspective view of one embodiment of an end-effector having scissors 440 for use with a rigidizable member 320. FIG. 20E is a perspective view one embodiment of an end-effector having a needle knife 450 for use with a rigidizable member 320. FIG. 20F is a perspective view of one embodiment of an end-effector having a sphincterotome 460 for use with a rigidizable member 320. FIG. 20G is a perspective view of one embodiment of an end-effector having a hook knife 470 for use with a rigidizable member 320.

The various embodiments of end-effectors discussed herein may be employed to perform various surgical procedures. A surgical apparatus is positioned in a patient. The surgical apparatus comprises a surgical instrument comprising a rigidizable member and an end-effector located at a distal end of the rigidizable member. The surgical instrument is inserted into a patient through an opening in the patient. An endoscope is inserted into the patient through the opening. The surgical instrument is positioned using the endoscope. The rigidizable member is rigidized using a rigidizing mechanism.

The devices disclosed herein can be designed to be disposed of after a single use, or they can be designed to be used multiple times. In either case, however, a device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present disclosure and appended claims.

Preferably, the various embodiments described herein will be processed before surgery. First, a new or used instrument is obtained and if necessary cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that the device is sterilized. This can be done by any number of ways known to those skilled in the art including beta or gamma radiation, ethylene oxide, and/or steam.

Although various embodiments have been described herein, many modifications and variations to those embodiments may be implemented. For example, different types of specimen retrieval bags and end-effectors may be employed. In addition, combinations of the described embodiments may be used. For example, the specimen retrieval bag may comprise a fused portion at the proximal end and an open portion at the distal end. Also, where materials are disclosed for certain components, other materials may be used. The foregoing description and following claims are intended to cover all such modification and variations.

Although the various embodiments of the rigidizable surgical instrument have been described herein in connection with certain disclosed embodiments, many modifications and variations to those embodiments may be implemented. For example, different types of end-effectors may be employed. Also, where materials are disclosed for certain components, other materials may be used. The foregoing description and following claims are intended to convey all such modifications and variations.

Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A surgical instrument, comprising:
a rigidizable member having a proximal end and a distal end;
a first collapsible arm located at the distal end of the rigidizable member;
a second collapsible arm located at the distal end of the rigidizable member;
a bag having an open end and a closed end, wherein the bag is configured to be retained upon the first collapsible arm and the second collapsible arm;
a central bore extending through the rigidizable member;
a rigidizing component disposed in the central bore, wherein the rigidizing component comprises a state-change material to rigidize the rigidizable member when the state-change material is in a rigid state;
wherein the rigidizable member is rendered substantially rigid when the rigidizing component is actuated; and
wherein the rigidizable member is rendered substantially flexible when the rigidizing component is deactuated.

2. A surgical instrument, comprising:
a rigidizable member having a proximal end and a distal end;
a first collapsible arm located at the distal end of the rigidizable member; and
a second collapsible arm located at the distal end of the rigidizable member.

3. The surgical instrument of claim 2, further comprising:
a central bore extending through the rigidizable member; and
a rigidizing component disposed in the central bore;
wherein the rigidizable member is rendered substantially rigid when the rigidizing component is actuated; and
wherein the rigidizable member is rendered substantially flexible when the rigidizing component is deactuated.

4. The surgical instrument of claim 3, wherein the rigidizing component comprises a tensioning wire to apply a tensioning force to rigidize the rigidizable member.

5. The surgical instrument of claim 3, wherein the rigidizing component comprises a state-change material to rigidize the rigidizable member when the state-change material is in a rigid state.

6. The surgical instrument of claim 2, further comprising:
a rigidizing mechanism coupled to the rigidizable member, wherein the rigidizable member is rendered substantially inflexible when the rigidizing mechanism actuates the rigidizing component and the rigidizable member is rendered substantially flexible when the rigidizing mechanism deactuates the rigidizing component.

7. The surgical instrument of claim 2, further comprising a flexible membrane disposed over the rigidizable member.

8. The surgical instrument of claim 2, wherein the rigidizable member comprises:
a socket; and
a ball partially inserted in the socket;
wherein adjacent surfaces of the ball and the socket coact and can rotate relative to each other in a flexible state; and
wherein the adjacent surfaces of the ball and socket are substantially locked in place when a tensioning force is applied to the ball and the socket.

9. The surgical instrument of claim 2, further comprising:
a hybrid shaft having a proximal end and a distal end, wherein the distal end is flexible, and wherein the proximal end is rigid; and
wherein the distal end of the hybrid shaft is connected to the proximal end of the rigidizable member.

10. The surgical instrument of claim 2, further comprising:
a bag having an open end and a closed end, wherein the bag is configured to be retained upon the first collapsible arm and the second collapsible arm;
a hybrid shaft having a proximal end and a distal end, wherein the distal end is flexible, and wherein the proximal end is rigid;
wherein the distal end of the hybrid shaft is connected to the proximal end of the rigidizable member;
a knot pusher located at the distal end of the rigidizable member;
an outer sheath extending from a distal handle to a distal end of the surgical instrument; and
a proximal handle.

11. The surgical instrument of claim 10, wherein the hybrid shaft extends from the proximal handle to the proximal end of the rigidizable member.

12. The surgical instrument of claim 10, wherein the outer sheath translates from an unfired position to a fired position upon translation of the distal handle towards the proximal handle.

13. The surgical instrument of claim 12, wherein the hybrid shaft, the first collapsible arm, the second collapsible arm, the bag, the rigidizable member, and the knot pusher are contained within the outer sheath in the unfired position.

14. The surgical instrument of claim 12, wherein the first collapsible arm, the second collapsible arm, the bag, the rigidizable member, and the knot pusher are removed from containment of the outer sheath in the fired position.

15. The surgical instrument of claim 14, wherein the outer sheath translates from the fired position to a retracted position upon translation of the distal handle from the proximal handle.

16. The surgical instrument of claim 15, wherein the first collapsible arm, the second collapsible arm, and the rigidizable member are contained within the outer sheath in the retracted position.

17. The surgical instrument of claim 16, comprising a suture extending from the proximal handle, through the knot pusher, through a top portion of the bag, and terminates with a knot at the knot pusher.

18. The surgical instrument of claim 17, wherein the suture is configured to close the bag upon pulling the suture at the proximal handle in the retracted position.

19. The surgical instrument of claim 17, wherein the knot pusher is configured to be retained at a distal end of the outer sheath in the retracted position.

20. The surgical instrument of claim 17, wherein the first collapsible arm and the second collapsible arm are rotatable.

21. The surgical instrument of claim 2, further comprising a bag having an open end and a closed end, wherein the bag is configured to be retained upon the first collapsible arm and the second collapsible arm.

22. The surgical instrument of claim 2, wherein the second collapsible arm extends distally beyond the first collapsible arm.

23. A method of positioning a surgical apparatus in a patient, the method comprising:
inserting a surgical instrument into a patient through an opening in the patient, the surgical instrument comprising a rigidizable member having a proximal end and a distal end, and an end-effector connected to the distal end of the rigidizable member;
inserting an endoscope into the patient through the opening in the patient;
positioning the surgical instrument with the endoscope; and
rigidizing the rigidzable member.

24. The method of claim 23, wherein the end-effector comprises a first collapsible arm, a second collapsible arm, and a bag having an open end and a closed end, wherein the bag is configured to be retained upon the first collapsible arm and the second collapsible arm; and wherein the surgical instrument further comprises a hybrid shaft having a proximal end and a distal end, wherein the distal end is flexible, and wherein the proximal end is rigid; wherein the distal end of the hybrid shaft is connected to the proximal end of the rigidizable member; a knot pusher located at the distal end of the rigidizable member; an outer sheath extending from a distal handle to a distal end of the surgical instrument; and a proximal handle, the method comprising:
translating the distal handle proximally to deploy the bag and the at least collapsible arm from the outer sheath;
receiving biological materials in the bag;
translating the distal handle distally to return the at least one collapsible arm to the outer sheath;
cinching the bag with the assistance of a knot pusher by pulling a suture at a proximal handle; and
removing the bag containing biological material from the patient.

25. The method of claim 23, comprising inserting the surgical instrument through an overtube.

26. The method of claim 25, comprising inserting the surgical instrument through an endoscope.

27. The method of claim 25, comprising inserting the surgical instrument between the overtube and the endoscope.

28. The method of claim 23, comprising inserting the surgical instrument adjacent to the endoscope.

29. A surgical instrument comprising:
a rigidizable member having a proximal end and a distal end; and
an end-effector connected to the distal end of the rigidizable member.

30. The surgical instrument of claim 29, further comprising a rigidizing component disposed in the rigidizable member, wherein the rigidizing component comprises a state-change material to rigidize the rigidizable member when the state-change material is in a rigid state; wherein the rigidizable member is rendered substantially rigid when the rigidizing component is actuated; and wherein the rigidizable member is rendered substantially flexible when the rigidizing component is deactuated.

31. The surgical instrument of claim 29, wherein the rigidizable member comprises a stiffening element configured to selectively stiffen when a vacuum force is applied thereto.

32. The surgical instrument of claim 29, wherein the end-effector comprises a bag.

33. The surgical instrument of claim 29, wherein the end-effector comprises grasper jaws.

34. The surgical instrument of claim 29, wherein the end-effector comprises biopsy jaws and a spike.

35. The surgical instrument of claim 29, wherein the end-effector comprises a snare loop.

36. The surgical instrument of claim 29, wherein the end-effector comprises scissors.

37. The surgical instrument of claim 29, wherein the end-effector comprises a needle knife.

38. The surgical instrument of claim 29, wherein the end-effector comprises a sphincterotome.

39. The surgical instrument of claim 29, wherein the end-effector comprises a hook knife.
